(19) United States
(12) Patent Application Publication
(10) Pub. No.: US 2020/0316229 A1
(43) Pub. Date: Oct. 8, 2020

(54) COMPOUNDS AND COMPOSITIONS USEFUL AS RADIOTRACERS FOR IMAGING OF REACTIVE OXIDATIVE SPECIES

(71) Applicant: Wake Forest University Health Sciences, Winston-Salem, NC (US)

(72) Inventor: Kiran K. Solingapuram Sai, Lewisville, NC (US)

(21) Appl. No.: 16/839,544

(22) Filed: Apr. 3, 2020

Related U.S. Application Data
(60) Provisional application No. 62/829,982, filed on Apr. 5, 2019.

(51) Int. Cl. A61K 51/04

(52) U.S. Cl. CPC A61K 51/0427

(57) ABSTRACT

Provided herein are compounds and compositions useful for imaging, detecting, and/or diagnosing oxidative stress and/or a ROS modulated illness by detection of gamma radiation emitted by the compound, as well as intermediate compounds and methods useful to make the compounds and/or compositions, and methods of use thereof.

### CROSS-REFERENCE TO RELATED APPLICATIONS

This application claims the benefit of U.S. Provisional Patent Application No. 62/829,982, filed Apr. 5, 2019, the disclosure of which is incorporate by reference herein in its entirety.

### BACKGROUND

Oxidative stress can be caused by an imbalance between the excess production of reactive oxygen species (ROS) and/or reactive nitrogen species (RNS) and their poor elimination by protective antioxidant mechanisms. Measuring ROS levels in vivo can allow clinicians to evaluate and guide treatment regimens for diseases, as oxidative stress is a common underlying factor for many types of cancers, neurodegenerative and cardiovascular disorders and leads to altered redox regulation of signaling pathways among several other deleterious effects.

Although multiple techniques/probes have been reported to track ROS, including electron spin resonance (ESR), fluorescent, chemiluminescent, and enzymatic probes, all have sensitivity and selectivity issues, thus limiting their use in a clinical environment. Hence, there is a need to develop probes that can be used in conjunction with clinical modalities to image oxidative stress level real-time in vivo.

### SUMMARY

Provided herein is a compound of Formula (I):

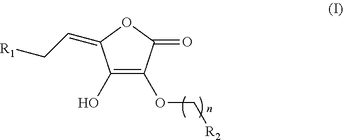

wherein:

R₁ is a substituted aryl or substituted heteroaryl, which is substituted with —X(CH₂)ₘ—R₃,

wherein X is O, S, NH, or CH₂;

m is 0, 1, 2, 3, 4, 5, or 6;

R₃ is:
(i) an imaging moiety selected from the group consisting of ¹⁸F, ⁷⁶Br, ¹²³I, Br, I, and ¹¹C;
(ii) a leaving group selected from the group consisting of a halo (e.g., Cl, F, Br and/or I) and a sulfonate (e.g., triflate, mesylate, tosylate, brosylate and/or nosylate); or
(iii) a chelator moiety (e.g., DOTA, NOTA and DFO) associated with a radioisotope selected from ⁶⁸Gd, ⁶⁴Cu, and ⁸⁹Zr;

n is 0; and

R₂ is H;

or wherein:

R₁ is halo (e.g., Cl, Br, F, I);

n is 0, 1, 2, 3, 4, 5, or 6; and

R₂ is a substituted aryl or substituted heteroaryl, which is substituted with —X(CH₂)ₘ—R₃,

wherein X is O, S, NH or CH₂;

m is 0, 1, 2, 3, 4, 5, or 6;

R₃ is:
(i) an imaging moiety selected from the group consisting of ¹⁸F, ⁷⁶Br, ¹²³I, and ¹¹C;
(ii) a leaving group selected from the group consisting of a halo (e.g., Cl, F, Br and/or I) and a sulfonate (e.g., triflate, mesylate, tosylate, brosylate and/or nosylate); or
(iii) a chelator moiety (e.g., DOTA, NOTA and DFO) associated with a radioisotope selected from ⁶⁸Gd, ⁶⁴Cu, and ⁸⁹Zr;
   or a pharmaceutically acceptable salt thereof.

In some embodiments, the compound is a compound of Formula (I)(a):

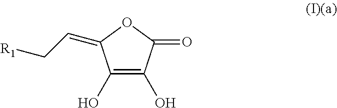

wherein:

R₁ is a substituted aryl or substituted heteroaryl, which is substituted with —X(CH₂)ₘ—R₃,

wherein X is O, S, NH or CH₂;

m is 0, 1, 2, 3, 4, 5, or 6; and

R₃ is: (i) an imaging moiety selected from the group consisting of ¹⁸F and ¹¹CH₃; or (ii) a leaving group selected from F and a sulfonate (e.g., a tosylate);

or a pharmaceutically acceptable salt thereof.

In some embodiments, the compound is a compound of Formula (I)(b):

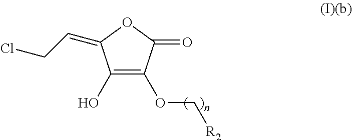

wherein

n is 1;

R₂ is a substituted aryl or substituted heteroaryl, which is substituted with —X(CH₂)ₘ—R₃,

wherein X is O, S, NH or CH₂;

m is 0, 1, 2, 3, 4, 5, or 6; and

R₃ is: (i) an imaging moiety selected from the group consisting of ¹⁸F and ¹¹CH₃; or (ii) a leaving group selected from the group consisting of F and a sulfonate (e.g., a tosylate);

or a pharmaceutically acceptable salt thereof.

In some embodiments, R₁ is a substituted aryl selected from anthracenyl, azulenyl, fluorenyl, indanyl, indenyl, naphthyl, phenyl, and tetrahydronaphthyl.

In some embodiments, R₂ is a substituted aryl selected from the group consisting of anthracenyl, azulenyl, fluorenyl, indanyl, indenyl, naphthyl, phenyl, and tetrahydronaphthyl. In some embodiments, the substituted aryl is a substituted phenyl.

In some embodiments, the substituted heteroaryl is selected from the group consisting of benzoxadiazolyl, benzoxazolyl, benzofuranyl, benzothienyl, furanyl, imidazolyl, indazolyl, indolyl, isoxazolyl, isoquinolinyl, isothiazolyl, naphthyridinyl, oxadiazolyl, oxazolyl, pyridinyl, pyridazinyl, pyrimidinyl, pyrazinyl, pyrazolyl, pyrrolyl, quinolinyl, thiazolyl, thienopyridinyl, thienyl, triazolyl, thiadiazolyl, and triazinyl.

In some embodiments, R₂ is a substituted heteroaryl selected from benzoxadiazolyl, benzoxazolyl, benzofuranyl, benzothienyl, furanyl, imidazolyl, indazolyl, indolyl, isoxazolyl, isoquinolinyl, isothiazolyl, naphthyridinyl, oxadiazolyl, oxazolyl, pyridinyl, pyridazinyl, pyrimidinyl, pyrazinyl, pyrazolyl, pyrrolyl, quinolinyl, thiazolyl, thienopyridinyl, thienyl, triazolyl, thiadiazolyl, and triazinyl; n is 1; and R₁ is Cl.

In some embodiments, the substituted heteroaryl is triazolyl, pyridinyl or pyrrolyl. In some embodiments, the X is O. In some embodiments, m is 2. In some embodiments, R₃ is ¹⁸F. In some embodiments, R₃ is halo (e.g., F). In some embodiments, R₃ is a sulfonate. In some embodiments, the R₃ is tosylate. In some embodiments, m is 0 and R₃ is ¹¹CH₃.

In some embodiments, R₃ is a ¹⁸F imaging moiety and exhibits a radiochemical specific activity from about 2500 to about 4000 mCi/μmol.

In some embodiments, the compound exhibits at least 80% serum stability for about 120 minutes post synthesis when contacting human serum.

Also provided is a method of measuring reactive oxygen species (ROS) in cells comprising: contacting cells with an effective amount of a compound as taught herein; and measuring gamma radiation emitted by the compound. In some embodiments, the ROS are measured in vitro. In some embodiments, the ROS are measured in vivo.

Further provided is a method of imaging oxidative stress in cells, comprising: contacting cells with an effective amount of a compound as taught herein; detecting gamma radiation emitted by the compound; and forming an image therefrom. In some embodiments, the cells are cancer cells. In some embodiments, the cells are cancer cells selected from head and neck squamous cell carcinoma, glioblastoma, breast cancer, and prostate cancer.

Also provided is a method of imaging/detecting/diagnosing a ROS modulated illness comprising: administering an effective amount of a compound of a compound as taught herein to a subject in need thereof; detecting gamma radiation emitted by the compound; and forming an image therefrom. In some embodiments, the ROS modulated illness is selected from diabetes, cardiovascular diseases, atherosclerosis, hypertension, ischemia, reperfusion injury, neurodegeneration, rheumatoid arthritis, and cancer.

Further provided is a method of making a compound of Formula (I)(b)(i):

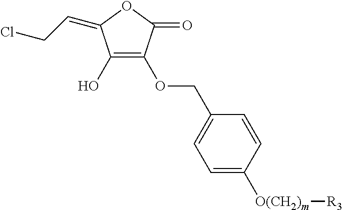

wherein:

m is 0 and R₃ is CH₃; or

m is 1, 2, 3, 4, 5 or 6, and R₃ is a leaving group (e.g., a sulfonate (e.g., tosylate, mesylate, brosylate, nosylate, triflate, besylate) or a halo),

comprising:

(a) providing a compound of Formula (A):

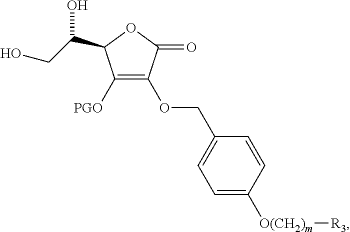

wherein PG is a hydroxyl protecting group, and m and R₃ are as defined above;

(b) contacting the compound of Formula (A) with a sulfonyl halide (e.g., tosyl chloride) to form a compound of Formula (B):

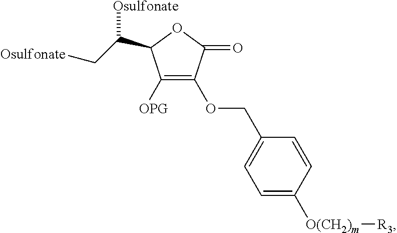

wherein PG, m and R₃ are as defined above;

(c) contacting the compound of Formula (B) with Et₄NCl and a base (e.g., DBU) to form a compound of Formula (C):

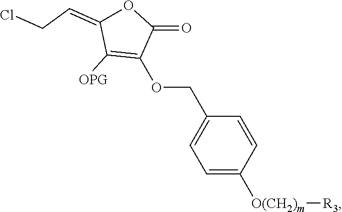

wherein PG, m and R₃ are the same as defined above;

(d) purifying the compound of Formula (C) to obtain the compound of Formula (C) with a chemical purity of at least 90%; and then

(e) removing the protecting group PG of the compound of Formula (C), to obtain the compound of Formula (I)(b)(i).

Also provided is a method of producing a radiotracer compound of Formula I:

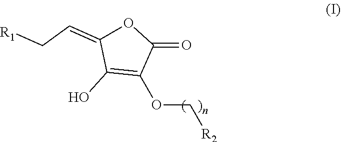

wherein:

R₁ is a substituted aryl or substituted heteroaryl, which is substituted with —X(CH₂)ₘ—R₃,

wherein X is O, NH, S or CH₂;

m is 0, 1, 2, 3, 4, 5, or 6;

R₃ is an ¹⁸F imaging moiety;

n is 0; and

R₂ is H;

or wherein:

R₁ is halo;

n is 0, 1, 2, 3, 4, 5, or 6; and

R₂ is a substituted aryl or substituted heteroaryl, which is substituted with —X(CH₂)ₘ—R₃,

wherein X is O, S, NH or CH₂;

m is 0, 1, 2, 3, 4, 5, or 6; and

R₃ is an ¹⁸F imaging moiety,

or a pharmaceutical acceptable salt thereof;
comprising:

(a) mixing a precursor compound of Formula (I);

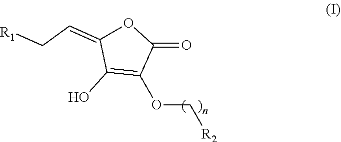

wherein R₁ is a substituted aryl or substituted heteroaryl, which is substituted with —X(CH₂)ₘ—R₃,
wherein X is O, NH, S or CH₂;

m is 0, 1, 2, 3, 4, 5, or 6;

R₃ is a leaving group;

n is 0; and

R₂ is H;

or wherein:

R₁ is halo;

n is 0, 1, 2, 3, 4, 5, or 6; and

R₂ is a substituted aryl or substituted heteroaryl, which is substituted with —X(CH₂)ₘ—R₃,

wherein X is O, S, NH or CH₂;

m is 0, 1, 2, 3, 4, 5, or 6;

R₃ is a leaving group selected from a sulfonate (e.g., tosylate) and halo,

with a solvent and a solution of [¹⁸F]F to obtain a reaction mixture;

(b) heating the reaction mixture obtained in step (a); and then

(c) purifying the reaction mixture, to produce the radiotracer compound.

Further provided is the use of a compound as taught herein in a method of imaging/detecting/diagnosing oxidative stress and/or a ROS modulated illness as taught herein in a subject (e.g., human subject).

### BRIEF DESCRIPTION OF THE DRAWINGS

FIG. 1A. Synthetic scheme of [¹⁸F]KS1 production.

FIG. 1B. Representative semiprep HPLC chromatogram with upper UV and lower radio γ trace of [¹⁸F]KS1.

FIG. 1C. QC spectrum of [¹⁸F]KS1 single injection (highlighted with arrow).

FIG. 1D. Injection of [¹⁸F]KS1 showed a single radioactive peak with minimal UV absorbance, indicating good specific activity.

FIG. 2. MitoSOX assay on of KS1 and KS2 with ascorbate as reference standard in SCC-61 and rSCC-61 cell lines, showing that lower concentrations of KS1 and KS2 detect ROS like ascorbate (***p<0.001).

FIG. 3. ROS specificity of KS1. Representative fluorescent images (left upper and lower panels) and lower intensity of KS1-mitoSOX in SCC-61 with SOD (1.0 mm) (**p<0.05).

FIG. 4. IC₅₀ values of KS1, KS2, and ascorbate (0.1 mM to 2.0 mM) in patient-derived cancer cell lines, showing that higher concentrations of KS1 and KS2 kills tumor cells like ascorbate (***p<0.001, **p<0.05).

FIG. 5. Cell uptake of [¹⁸F]KS1 in HNSCC at baseline, promoter and blockade conditions (n=6) after 60 min in vitro. The data were expressed as % injected dose (ID)/mg of protein present in each well. Plates without ligands are controls.

FIG. 6. PC3 cell uptake of [¹⁸F]KS1 at baseline, promoter and blockade conditions (n=6) under normoxic and hypoxic conditions after 60 min. The data were expressed as % injected dose (ID)/mg of protein present in each well. Plates without ligands were controls.

FIG. 7. Ex vivo serum stability of [¹⁸F]KS1 and [¹⁸F]KS2.

FIG. 8. Representative PET images of [¹⁸F]KS1 in PC3-bearing mice (n=3). Panel A is baseline and Panel B is blocking with KS1 with arrow mark highlighting tumor (T). Also seen are primary excretory organs as bladder and kidneys.

FIG. 9. Standard biodistribution of [¹⁸F]KS1 in PC3 bearing mice (n=3), with tumor. Muscle ratio highlighted in the inset box.

FIG. 10. DCFDA assay of KS1 and ascorbate at 10 and 100 μM concentration in SCC-61 and PC3 cell lines (***p=0.005).

FIG. 11. DCFDA assay of KS1 and ascorbate at 2 mM concentration in SCC-61 and PC3 cell lines (****p≤0.001).

FIG. 12. IC₅₀ values of KS1 and ascorbate in multiple patient derived tumor cell lines with **p=0.01.

FIG. 13. Cell uptake of [¹⁸F]KS1 at baseline, promoter and blockade conditions (n=6) in rSCC-61 and SCC-61 cell lines. The data were expressed as % injected dose (ID)/mg of protein present in each well, with ***p≤0.005.

FIG. 14. Representative A. sagittal and B. axial mPET/CT images of [¹⁸F]KS1 in (1) SCC61 (2) rSCC-61 tumor-bearing mice (n=3) and (3) avg tumor ROI-based uptake from the PET images, **p<0.01.

FIG. 15. Standard biodistribution of [¹⁸F]KS1 in normal mice, (n=8: 4 male and 4 female mice), ***p≤0.001.

FIG. 16. Standard biodistribution of [¹⁸F]KS1 in SCC-61 and rSCC-61 tumor-bearing mice (n=4), and with tumor: muscle ratio highlighted in the box; ***p≤0.001.

### DETAILED DESCRIPTION

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant invention. Hence, the following specification is intended to illustrate some particular embodiments of the invention, and not to exhaustively specify all permutations, combinations and variations thereof.

The disclosures of all patent references cited herein are hereby incorporated by reference to the extent they are consistent with the disclosure set forth herein. As used herein in the description of the invention and the appended claims, the singular forms “a,” “an” and “the” are intended to include the plural forms as well, unless the context clearly indicates otherwise.

Subjects, tissues, cells, cell fractions, and proteins utilized to carry out the present invention may be of any suitable source including microbial (including gram negative and gram positive bacteria, yeast, algae, fungi, protozoa, and viral, etc.), plant (including both monocots and dicots) and animal (including mammalian, avian, reptile, and amphibian species, etc.). Mammalian subjects include both humans and other animal species treated for veterinary purposes (including but not limited to monkeys, dogs, cats, cattle, horses, sheep, rats, mice, rabbits, goats, etc.).

As used herein in the accompanying chemical structures, “H” refers to a hydrogen atom. “C” refers to a carbon atom. “N” refers to a nitrogen atom. “S” refers to a sulfur atom. “O” refers to an oxygen atom.

“Alkyl,” as used herein, refers to a saturated straight or branched chain, or cyclic hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, cyclopropyl, cyclobutyl, cyclopentyl, cyclohexyl, and the like. “Lower alkyl” as used herein, is a subset of alkyl and refers to a straight or branched chain hydrocarbon group containing from 1 to 4 carbon atoms. Representative examples of lower alkyl include, but are not limited to, methyl, ethyl, n-propyl, iso-propyl, n-butyl, iso-butyl, tert-butyl, cyclopropyl, cyclobutyl, and the like. The alkyl groups may be optionally substituted with one or more suitable substituents, such as halo, hydroxy, carboxy, amine, etc.

“Aryl,” as used herein, refers to a monocyclic carbocyclic ring system or a bicyclic carbocyclic fused or directly adjoining ring system having one or more aromatic rings. Examples include, but are not limited to, phenyl, indanyl, indenyl, tetrahydronaphthyl, and the like. As noted, in some embodiments, the aryl has two aromatic rings, which rings are fused or directly adjoining. Examples include, but are not limited to, biphenyl, napthyl, azulenyl, etc. The aryl may be optionally substituted with one or more suitable substituents, such as alkyl, halo, hydroxy, carboxy, amine, etc. In some embodiments, the aryl may be substituted with —X(CH₂)ₘ—R₃, wherein X is O, NH, S or CH₂; m is an integer selected from 0, 1, 2, 3, 4, 5, and 6 (e.g., 1, 2, or 3); and R₃ is: (i) an imaging moiety selected from ¹⁸F, ⁷⁶Br, ¹²³I, and ¹¹C, (ii) a leaving group selected from a halo (Cl, F, Br or I) and a sulfonate (e.g., triflate, mesylate, tosylate, brosylate and/or nosylate), or (iii) a chelator molecule (e.g., DOTA, NOTA and DFO) associated with a radioisotope selected from ⁶⁸Gd, ⁶⁴Cu, and ⁸⁹Zr.

“Heteroaryl,” as used herein, refers to a monovalent aromatic group having a single ring or two fused or directly adjoining rings and containing in at least one of the rings at least one heteroatom (typically 1 to 3) independently selected from nitrogen, oxygen and sulfur. Examples include, but are not limited to, pyrrole, imidazole, thiazole, oxazole, furan, thiophene, triazole, pyrazole, isoxazole, isothiazole, pyridine, pyrazine, pyridazine, pyrimidine, triazine, and the like. As noted, in some embodiments, the heteroaryl has two aromatic rings, which rings are fused or directly adjoining. Examples include, but are not limited to, benzothiophene, benzofuran, indole, benzimidazole, benzothiazole, quinoline, isoquinoline, quinazoline, quinoxaline, phenyl-pyrrole, phenyl-thiophene, etc. The heteroaryl may be optionally substituted with one or more suitable substituents, such as alkyl, halo, hydroxy, carboxy, amine, etc. In some embodiments, the aryl may be substituted with —X(CH₂)ₘ—R₃, wherein X is O, NH, S or CH₂; m is an integer selected from 0, 1, 2, 3, 4, 5, and 6 (e.g., 1, 2, or 3); and R₃ is: (i) an imaging moiety selected from ¹⁸F, ⁷⁶Br, ¹²³I, and ¹¹C, (ii) a leaving group selected from a halo (Cl, F, Br or I) and a sulfonate (e.g., triflate, mesylate, tosylate, brosylate and/or nosylate), or (iii) a chelator molecule (e.g., DOTA, NOTA and DFO) associated with a radioisotope selected from ⁶⁸Gd, ⁶⁴Cu, and ⁸⁹Zr.

The terms “halo” and “halogen,” as used herein, refer to fluoro (—F), chloro (—Cl), bromo (—Br), or iodo (—I).

The term “chelator” as used herein, refers to a moiety or group that can bind to a metal ion through one or more donor atoms. Example chelators include, but are not limited to, 1,4,7,10-Tetraazacyclododecane-1,4,7,10-tetraacetic acid (DOTA), 1,4,7-triazacyclononane-N,N′,N″-triacetic acid (NOTA) and/or N′-[5-(Acetyl-hydroxy-amino)pentyl]-N-[5-[3-(5-aminopentyl-hydroxy-carbamoyl) propanoylamino]pentyl]-N-hydroxy-butane diamide (DFO). See, e.g., U.S. Pat. Nos. 4,639,365 and 8,153,101.

“Imaging moiety” as used herein may be any suitable detectable or otherwise functional group that enables imaging, including but not limited to radioactive isotopes, which may emit gamma radiation detectable with a suitable detector such as for positron emission tomography (PET) imaging, and groups and/or chelating groups containing the same. PET compatible radioisotopes include, but are not limited to, fluorine-18, carbon-11, gallium-68, zirconium-89, copper-64, nitrogen-13, iodine-123, iodine-125, gallium-68, etc. In some embodiments, fluorine-18 or carbon-11 is preferred.

A “leaving group” is a group or substituent of a compound that can be displaced by another group or substituent in a substitution reaction, such as a nucleophilic substitution reaction. For example, common leaving groups include halogens, and sulfonate leaving groups, such as a mesylate (methane sulfonate or —OMs), tosylate (p-toluenesulfonate or —OTs), brosylate, nosylate, besylate (benzene sulfonate). See, e.g., U.S. Pat. No. 8,101,643.

As used herein, the phrase “pharmaceutically acceptable” refers to those compounds, materials, compositions, and/or dosage forms that are, within the scope of sound medical judgment, suitable for use in contact with the tissues of human beings and animals without excessive toxicity, irritation, allergic response, or other problem or complication, commensurate with a reasonable benefit/risk ratio.

A “diagnostic kit” or “kit” comprises a collection of components, termed the formulation, in one or more vials which are used by the practicing end user in a clinical or pharmacy setting to synthesize radiopharmaceuticals useful for diagnostic procedures. The kit preferably provides all the requisite components to synthesize and use the diagnostic pharmaceutical except those that are commonly available to the practicing end user, such as water or saline for injection, a solution of the radionuclide, equipment for heating the kit during the synthesis of the radiopharmaceutical, if required, equipment necessary for administering the radiopharmaceutical to the patient such as syringes, shielding, imaging equipment, and the like. Imaging agents are provided to the end user in their final form in a formulation contained typically in one vial, as either a solid (e.g., lyophilized, amorphous, or crystalline) or an aqueous solution. The end user typically reconstitutes the lyophilized material with water or saline and withdraws the patient dose or just withdraws the dose from the aqueous solution formulation as provided.

Ascorbate (vitamin C, ascorbic acid) is known as a potent biological antioxidant that scavenges reactive oxygen species (ROS) and reactive nitrogen species (RNS) that could otherwise damage nucleic acids and promote carcinogenesis. Oxidative stress is a common underlying factor for many types of cancers, neurodegenerative and cardiovascular disorders and may lead to altered redox regulation of signaling pathways among several other deleterious effects. See, e.g., Uttara et al., “Oxidative Stress and Neurodegenerative Diseases: A Review of Upstream and Downstream Antioxidant Therapeutic Options,” Current Neuropharmacology 2009, 7, 65-74; Sosa et al., “Oxidative stress and cancer: An overview,” Ageing Research Reviews, 2013, 12(1), 376-390; Reuter et al., “Oxidative stress, inflammation, and cancer: How are they linked?” Free Redic Biol Med. 2010, 49(11), 1603-1616; Noda et al., “Cancer and Oxidative Stress,” JMAJ 2011, 44(12), 535-539.

### I. Active Compounds.

The present invention provides compounds of Formula I:

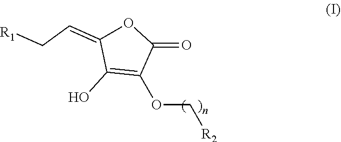

wherein:

R₁ is a substituted aryl or substituted heteroaryl, which is substituted with —X(CH₂)ₘ—R₃,

wherein X is O, NH, S or CH₂;

m is 0, 1, 2, 3, 4, 5, or 6;

R₃ is:
(i) an imaging moiety selected from the group consisting of ¹⁸F, ⁷⁶Br, ¹²³I, and ¹¹C;
(ii) a leaving group selected from the group consisting of a halo (e.g., Cl, F, Br and/or I) and a sulfonate (e.g., triflate, mesylate, tosylate, brosylate and/or nosylate); or
(iii) a chelator moiety (e.g., DOTA, NOTA and DFO) associated with a radioisotope selected from ⁶⁸Gd, ⁶⁴Cu, and ⁸⁹Zr;

n is 0; and

R₂ is H;

or wherein:

R₁ is halo (e.g., Cl, Br, F, I);

n is 0, 1, 2, 3, 4, 5, or 6; and

R₂ is a substituted aryl or substituted heteroaryl, which is substituted with —X(CH₂)ₘ—R₃,

wherein X is O, S, NH or CH₂;

m is 0, 1, 2, 3, 4, 5, or 6;

R₃ is:
(i) an imaging moiety selected from the group consisting of ¹⁸F, ⁷⁶Br, ¹²³I; and ¹¹C;
(ii) a leaving group selected from the group consisting of a halo (e.g., Cl, F, Br and/or I) and a sulfonate (e.g., triflate, mesylate, tosylate, brosylate and/or nosylate); or
(iii) a chelator moiety (e.g., DOTA, NOTA and DFO) associated with a radioisotope selected from ⁶⁸Gd, ⁶⁴Cu, and ⁸⁹Zr;
   or a pharmaceutically acceptable salt thereof.

In some embodiments, R₁ and/or R₂ are a substituted aryl independently selected from anthracenyl, azulenyl, fluorenyl, indanyl, indenyl, naphthyl, phenyl, and tetrahydronaphthyl. In some embodiments, R₁ is a substituted phenyl. In some embodiments, R₂ is a substituted phenyl.

In some embodiments, R₁ and/or R₂ are a substituted heteroaryl independently selected from benzoxadiazolyl, benzoxazolyl, benzofuranyl, benzothienyl, furanyl, imidazolyl, indazolyl, indolyl, isoxazolyl, isoquinolinyl, isothiazolyl, naphthyridinyl, oxadiazolyl, oxazolyl, pyridinyl, pyridazinyl, pyrimidinyl, pyrazinyl, pyrazolyl, pyrrolyl, quinolinyl, thiazolyl, thienopyridinyl, thienyl, triazolyl, thiadiazolyl, and triazinyl. In some embodiments, R₁ is a substituted triazolyl, pyridinyl or pyrrolyl. In some embodiments, R₂ is a substituted triazolyl, pyridinyl or pyrrolyl.

In some embodiments, R₃ is an imaging moiety. In some embodiments, R₃ is a ¹⁸F or ¹¹C imaging moiety, wherein the ¹¹C imaging moiety is selected from ¹¹CH₃, ¹¹CN, and ¹¹COOH.

In some embodiments, R₃ is a leaving group. In some embodiments, R₃ is selected from F or a sulfonate. In some embodiments, R₃ is a tosylate. In some embodiments, R₃ is halo (e.g., F).

In some embodiments, the compound of the invention is a compound of Formula (I)(a):

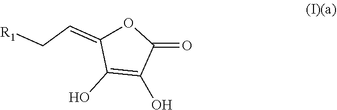

wherein:

R₁ is a substituted aryl or substituted heteroaryl, which is substituted with —X(CH₂)ₘ—R₃ as described above;

or a pharmaceutically salt thereof.

In some embodiments, R₁ is a substituted phenyl. In some embodiments, R₁ is a substituted triazolyl, pyridinyl or pyrrolyl. In some embodiments, X is O. In some embodiments, m is 2. In some embodiments, R₃ is an ¹⁸F imaging moiety. In some embodiments, R₃ is F. In some embodiments, R₃ is a sulfonate leaving group. In some embodiments, R₃ is tosylate.

In some embodiments, R₁ is a substituted phenyl and X is O. In some embodiments, R₁ is a substituted phenyl; X is O and m is 2. In some embodiments, R₁ is a substituted phenyl; X is O; m is 2 and R₃ is an ¹⁸F imaging moiety. In some embodiments, R₁ is a substituted phenyl; X is O; m is 2 and R₃ is a leaving group. In some embodiments, R₁ is a substituted phenyl; X is O; m is 2 and R₃ is F. In some embodiments, R₁ is a substituted phenyl; X is O; m is 2 and R₃ is a sulfonate leaving group. In some embodiments, R₁ is a substituted phenyl; X is O; m is 2 and R₃ is tosylate.

In some embodiments, m is 0 and R₃ is a ¹¹C imaging moiety. In some embodiments, m is 0 and R₃ is a ¹¹CH₃ imaging moiety.

In some embodiments, the compound of Formula (I)(a) is selected from the group consisting of:

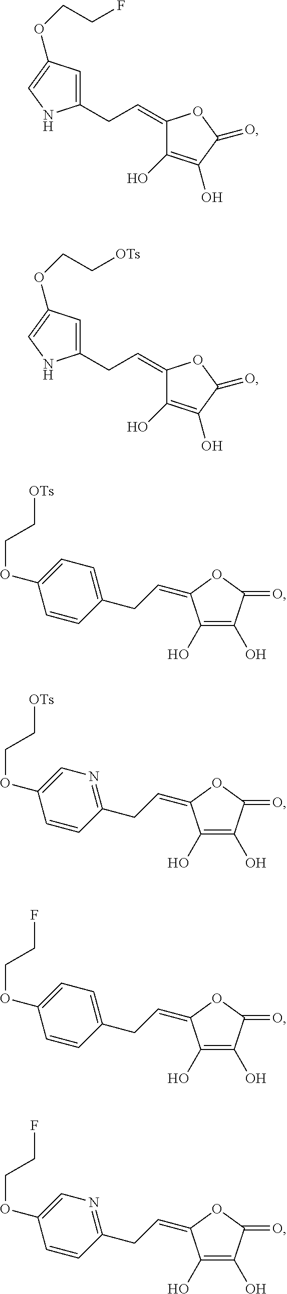 [SMILES: O=C1O/C(=C/Cc2cc(OCCF)c[nH]2)C(O)=C1O ; Cc1ccc(S(=O)(=O)OCCOc2c[nH]c(C/C=C3/OC(=O)C(O)=C3O)c2)cc1 ; Cc1ccc(S(=O)(=O)OCCOc2ccc(C/C=C3/OC(=O)C(O)=C3O)cc2)cc1 ; Cc1ccc(S(=O)(=O)OCCOc2ccc(C/C=C3/OC(=O)C(O)=C3O)nc2)cc1 ; O=C1O/C(=C/Cc2ccc(OCCF)cc2)C(O)=C1O ; O=C1O/C(=C/Cc2ccc(OCCF)cn2)C(O)=C1O]

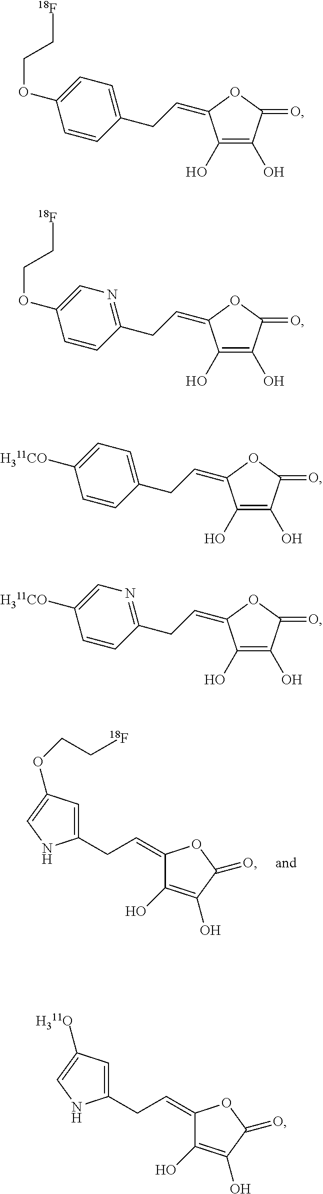

or a pharmaceutically acceptable salt thereof.

In some embodiments, the compound of the invention is a compound of Formula (I)(b):

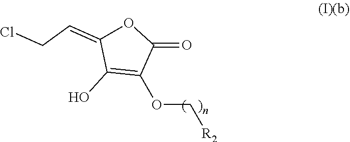

wherein:

R₂ is a substituted aryl or substituted heteroaryl, which is substituted with —X(CH₂)ₘ—R₃ as described above;

or a pharmaceutically acceptable salt thereof.

In some embodiments, R₂ is a substituted phenyl and X is O. In some embodiments, R₂ is a substituted phenyl; X is O and m is 2. In some embodiments, R₂ is a substituted phenyl; X is O; m is 2 and R₃ is an ¹⁸F imaging moiety. In some embodiments, R₂ is a substituted phenyl; X is O; m is 2 and R₃ is a leaving group. In some embodiments, R₂ is a substituted phenyl; X is O; m is 2 and R₃ is F. In some embodiments, R₂ is a substituted phenyl; X is O; m is 2 and R₃ is a sulfonate leaving group. In some embodiments, R₂ is a substituted phenyl; X is O; m is 2 and R₃ is a tosylate.

In some embodiments, m is 0 and R₃ is a ¹¹C imaging moiety. In some embodiments, m is 0 and R₃ is a ¹¹CH₃ imaging moiety.

In some embodiments, the compound of Formula (I)(b) is selected from the group consisting of:

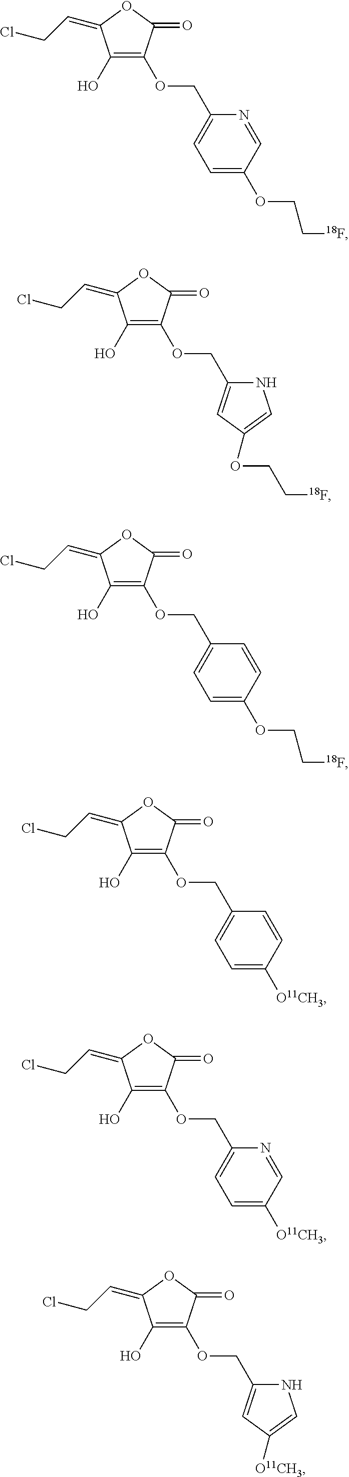

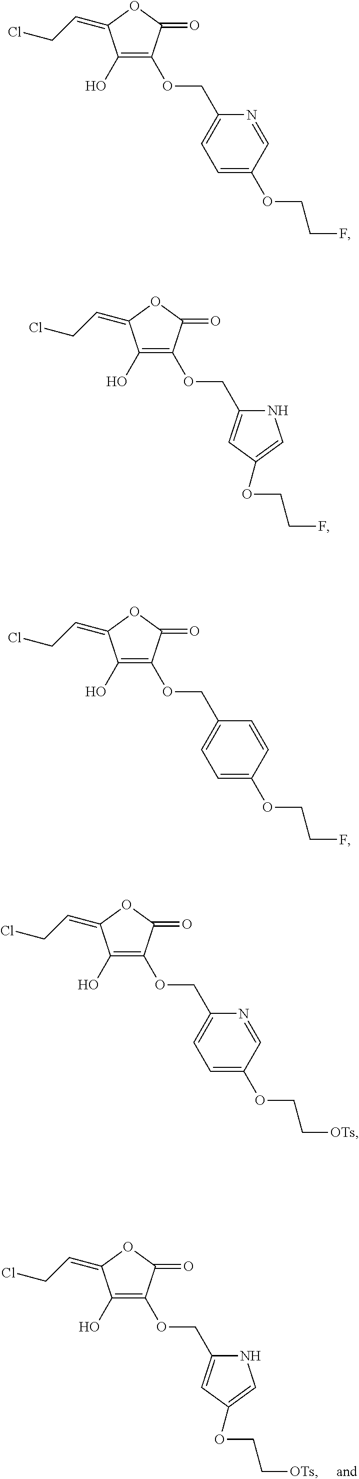 [SMILES: O=C1O/C(=C/CCl)C(O)=C1OCc1ccc(OCCF)cn1 ; O=C1O/C(=C/CCl)C(O)=C1OCc1cc(OCCF)c[nH]1 ; O=C1O/C(=C/CCl)C(O)=C1OCc1ccc(OCCF)cc1 ; Cc1ccc(S(=O)(=O)OCCOc2ccc(COC3=C(O)/C(=C\CCl)OC3=O)nc2)cc1 ; Cc1ccc(S(=O)(=O)OCCOc2c[nH]c(COC3=C(O)/C(=C\CCl)OC3=O)c2)cc1]

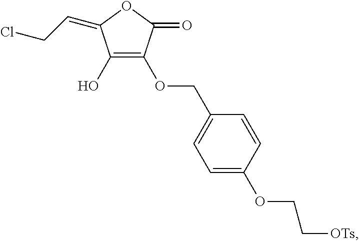 [SMILES: Cc1ccc(S(=O)(=O)OCCOc2ccc(COC3=C(O)/C(=C\CCl)OC3=O)cc2)cc1]

or a pharmaceutically acceptable salt thereof.

In some embodiments, the compounds of the invention, such as the compounds of Formula (I), (I)(a), and/or (I)(b) exhibit a radiochemical specific activity ranging from about 2500 to about 4000 μCi/mmol, from about 2700 to about 3850 μCi/mmol, or from about 3200 to about 3850 μCi/mmol (or at least about 2500, about 2600, about 2700, about 2800, about 2900, about 3000, about 3100, about 3200, about 3300, about 3400, about 3500, about 3600, about 3700, or at least about 3800 μCi/mmol). In some embodiments, compounds exhibiting such a radiochemical specific activity have an ¹⁸F imaging moiety as R₃.

In some embodiments, the compounds of the invention, particularly compounds of the invention with an imaging moiety, exhibit a good serum stability over a given time period. For example, in some embodiments, compounds of the invention with an ¹⁸F imaging moiety exhibit a serum stability of at least 80% after about 30 minutes, about 60 minutes, about 90 minutes, about 120 minutes, about 180 minutes or about 120 minutes after being exposed to and/or in contact with human serum ex vivo.

Pharmaceutically acceptable salts are salts that retain the desired activity of the parent compound and do not impart undesired toxicological effects. Examples of such salts are (a) acid addition salts formed with inorganic acids, for example hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (b) salts formed from elemental anions such as chlorine, bromine, and iodine.

### II. Methods of Synthesis.

Methods of synthesizing compounds as taught herein may be carried out as exemplified below or in accordance with methods known to those skilled in the art.

One aspect of the invention relates to a method of making a compound of Formula (I)(b)(i):

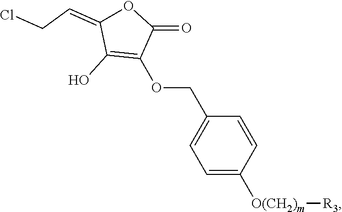

wherein

m is 0 and R₃ is CH₃; or

m is 1, 2, 3, 4, 5 or 6 and R₃ is a leaving group (e.g., a sulfonate (e.g., tosylate, mesylate, brosylate, nosylate, triflate, besylate) or a halo),

comprising:

(a) providing a compound of Formula (A):

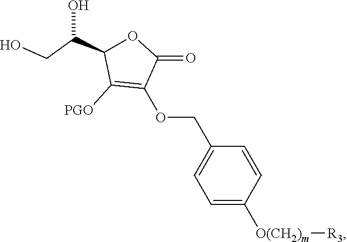

wherein

PG is a hydroxyl protecting group; and

m and R₃ are as defined above;

(b) contacting the compound of Formula (A) with a sulfonyl halide (e.g., tosyl chloride) to form a compound of Formula (B):

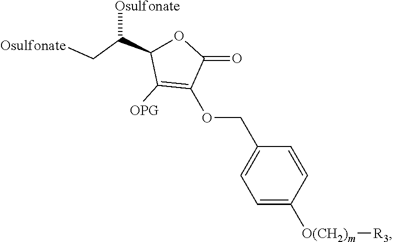

wherein

PG, m and R₃ are as defined above;

(c) contacting the compound of Formula (B) with Et₄NCl and a base (e.g., DBU) to form a compound of Formula (C):

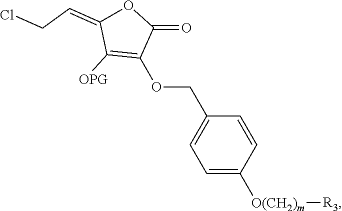

wherein

PG, m and R₃ are the same as described above;

(d) purifying the compound of Formula (C) to obtain the compound of Formula (C) with a chemical purity of at least 90%; and then

(e) removing the protecting group PG of the compound of Formula (C), to obtain the compound of Formula (I)(b)(i).

In some embodiments, the method further comprises purification of the compound of Formula (A) and/or of the compound of Formula (B) to obtain a diastereomeric excess (de) of from about 70% to about 100%, about 80% to about 100%, about 90% to about 100% (or at least 70%, 75%, 80%, 85%, 90%, 95%, 96%, 97%, 98%, 99%, 99.5% de) of the compound.

Purification of compounds of Formula (A), (B), and/or (C) may employ suitable methods known in the art such as, but not limited to, column chromatography (e.g., silica gel, alumina), reverse-phase chromatography, high pressure liquid chromatography (HPLC), distillation, liquid-liquid extraction, crystallization, etc., including combinations thereof.

The contacting step (b) may be carried out using methods known in the art. For example, in some embodiments, the compound of Formula (A) may be contacted with a sulfonyl halide in the presence of a base such as, but not limited to, pyridine, dimethylamino pyridine (DMAP), or a combination thereof. In some embodiments, the compound is contacted with a sulfonyl halide in the presence of pyridine. In some embodiments, the sulfonyl halide is a sulfonyl chloride, sulfonyl bromide, or sulfonyl iodide. Exemplary sulfonyl halides include, but are not limited to, mesyl chloride, chloro triflate, tosyl chloride, brosyl bromide, nosyl bromide, besyl chloride, or a combination thereof. In some embodiments, the intermediate of step (a) is contacted with a tosyl chloride in the presence of a base (e.g., pyridine).

The base employed in contacting step (c) together with Et₄NCl can be any suitable base known in the art. For example, in some embodiments, the compound of Formula (B) of step (b) is contacted with Et₄NCl in the presence of DBU (1,8-diazabicyclo[5.4.0]undec-7-ene).

The hydroxyl protecting group PG may be any suitable hydroxyl protecting group known in the art. In some embodiments, the hydroxyl protecting group is a benzyl group. Conditions to remove the hydroxyl protecting group depends upon the type of hydroxyl protecting group being employed, and a skilled artisan would be aware of the different reaction conditions required to remove various hydroxyl protecting groups. For example, in some embodiments, the benzyl protecting group is removed in the presence of Pb(OAc)₄.

In some embodiments, the compounds synthesized by the method of the invention are compounds of Formula (D), wherein m is 2 and R₃ is a leaving group. For example, in some embodiments, R₃ is F. In some embodiments, R₃ is a tosylate. Examples:

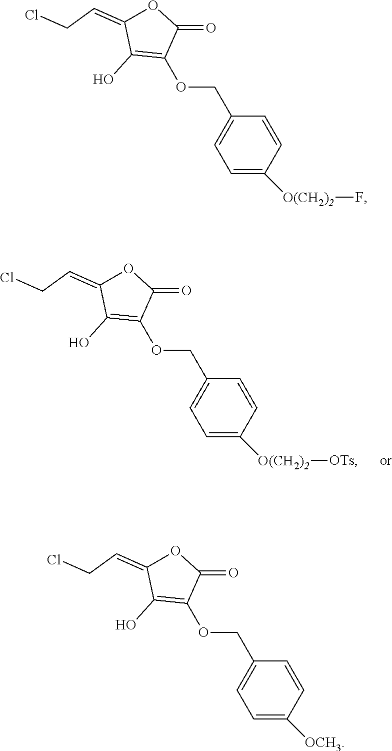

An example of the synthetic method of the invention is shown below. Briefly, 5¹, 6¹-dihydroxyl groups of L-ascorbic acid (1) are protected by acetone/acetyl chloride to obtain a ketal intermediate, 2. The ketal intermediate 2 was then reacted with 4¹-bromobenzyl substituted ethoxy alkanes, followed by acid deprotection to give 2¹-benzyloxy, 5¹, 6¹-dihydroxyl alkyl substituted ascorbic acid, 3. Dehydration and selective 3¹-debenzylation of the isolated product from the previous step 3 resulted in KS1. The corresponding F-18 radiolabeling precursor unit KS-OTs was prepared using the ethoxy tosyl substituted alkanes with intermediate 2, followed by deprotection and dehydration steps.

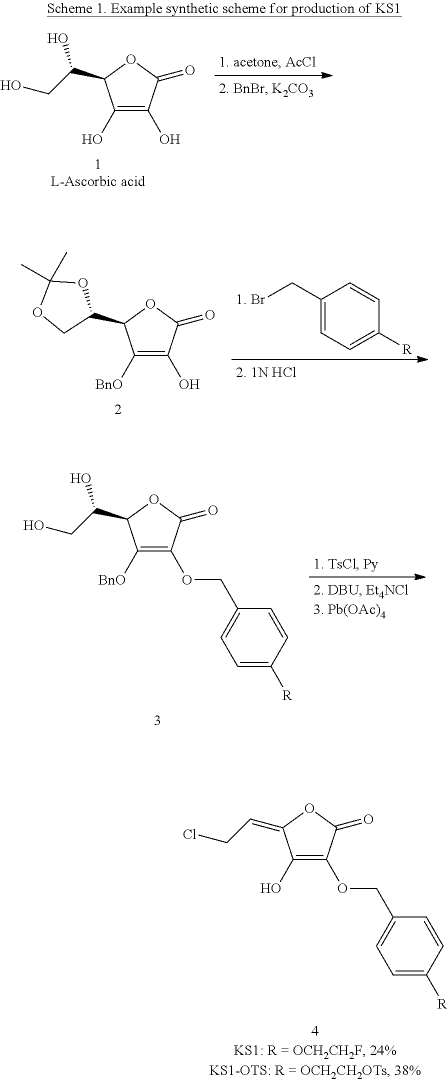

Another aspect of the invention relates to methods for synthesizing compounds of Formula (I)(a)(i):

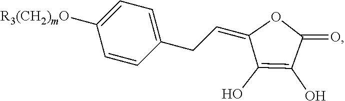

wherein:

m is 0 and R₃ is CH₃; or

m is 1, 2, 3, 4, 5, or 6 and R₃ is a leaving group (e.g., a sulfonate (e.g., tosylate, mesylate, brosylate, nosylate, triflate, besylate) or a halo).

In some embodiments, the compounds synthesized by the method of the invention comprise compounds of Formula (I)(a)(i), wherein m is 2 and R₃ is a leaving group. In some embodiments, R₃ is a tosylate. In some embodiments, R₃ is a halo (e.g., F).

In some embodiments, the compound of Formula (I)(a)(i) is:

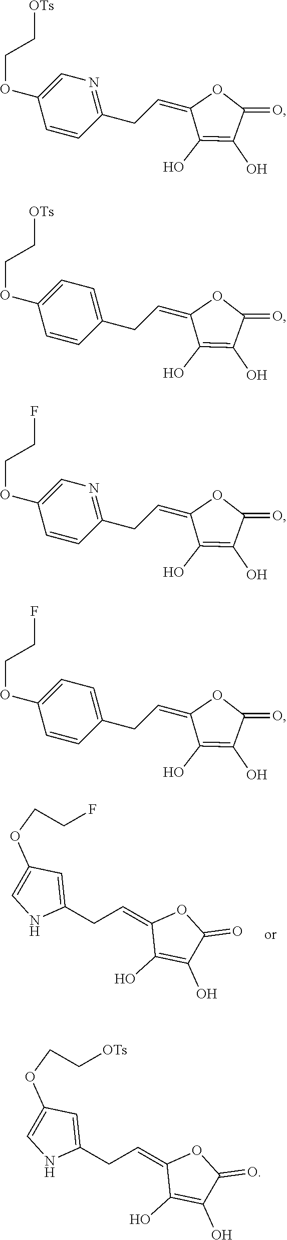 [SMILES: Cc1ccc(S(=O)(=O)OCCOc2ccc(C/C=C3/OC(=O)C(O)=C3O)nc2)cc1 ; Cc1ccc(S(=O)(=O)OCCOc2ccc(C/C=C3/OC(=O)C(O)=C3O)cc2)cc1 ; O=C1O/C(=C/Cc2ccc(OCCF)cn2)C(O)=C1O ; O=C1O/C(=C/Cc2ccc(OCCF)cc2)C(O)=C1O ; O=C1O/C(=C/Cc2cc(OCCF)c[nH]2)C(O)=C1O ; Cc1ccc(S(=O)(=O)OCCOc2c[nH]c(C/C=C3/OC(=O)C(O)=C3O)c2)cc1]

A skilled artisan would recognize that multiple synthetic pathways are possible to prepare a compound of formula (I)(a)(i) and thus, the current invention is not limited to any particular method of synthesizing a compound of Formula (I)(a)(i).

An example synthetic pathway of the synthetic method of the invention is shown below in Scheme 2.

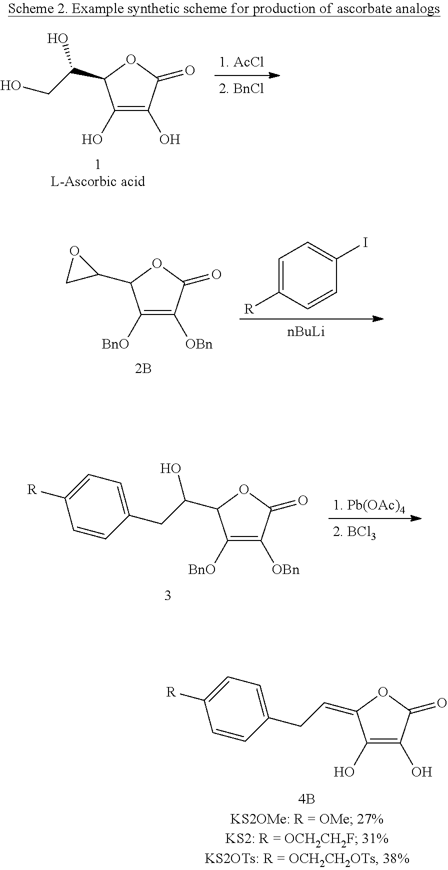

In this example synthesis, both hydroxy groups of the ascorbate core structure were maintained and groups at the 6′ position were modified to enable radiolabeling. 6′ aryl substituted L-ascorbic acid KS2-OMe and KS2 were prepared as depicted in Scheme 2. Briefly, L ascorbic acid (1) underwent epoxidation and benzylation to form a di-O-benzyl substituted epoxide intermediate, 3,4-bis(benzyloxy)-5-(oxiran-2-yl)furan-2(5H)-one, 2B. When subjected to ring opening reaction with 4-aryl iodide, this epoxide produced an aryl substituted furanose derivative, 3B. After dehydration and debenzylation reactions the results were the final compounds, KS2-OMe (with p-methoxyphenyl), and KS2 (with 2′ethoxyfluoro base) in 27% and 31% isolated chemical yields respectively. Simultaneously, the corresponding [¹⁸F]-tosyl precursor KS2-OTs was also synthesized by similar methods (38% chemical yield). All intermediates and final compounds may be characterized using ¹H-NMR and high resolution mass spectrometry.

Compounds as taught herein are useful as radiotracers or intermediates useful to create radiotracers containing an imaging moiety with a radioisotope selected from ¹⁸F, ⁷⁶Br, ¹²³I, and ¹¹C or containing a chelator molecule (e.g., DOTA, NOTA and DFO) associated with a radioisotope selected from ⁶⁸Gd, ⁶⁴Cu, and ⁸⁹Zr. In compounds of the invention with an imaging moiety containing a radioisotope selected from ¹⁸F, ⁷⁶Br, ¹²³I, and ¹¹C, the radioisotope is directly bound via a covalent bound to an atom (e.g., carbon or oxygen) to the backbone of the ascorbate structure. In compounds of the invention wherein the imaging moiety contains a chelator molecule (e.g., DOTA, NOTA and DFO) associated with a radioisotope selected from ⁶⁸Gd, ⁶⁴Cu, and ⁸⁹Zr, it is the chelator moiety that is directly bound to the backbone of the ascorbate structure via a covalent bound from one atom of the ascorbate backbone structure to one atom of the chelator molecule. See, e.g., Wadas et al., “Coordinating Radiometals of Copper, Gallium, Indium, Yttrium, and Zirconium for PET and SPECT Imaging of Disease,” Chem Rev 2010, 110, 2858-2902; Price et al., “Matching chelators to radiometals for radiopharmaceuticals,” Chemical Society Reviews 2014, 43(1), 260-290; Tsionou et al., “Comparison of macrocyclic and acyclic chelators for gallium-68 radiolabelling,” RSC Advcances 2017, 7, 49586-49599. See also US 2008/0267882 to Chen et al.; and WO 2005/087275 to Chao et al., which are incorporated by reference herein.

One aspect of the invention relates to a method of synthesizing compounds of the invention containing an imaging moiety with a fluorine-18 (¹⁸F) radioisotope. Typically, 18F labeled compounds are synthesized by S_{N}2 displacement of an appropriate leaving group. These leaving groups may be sulfonates such as toluenesulfonate (tosylate, TsO), methanesulfonate (mesylate, MsO), or trifluoromethanesulfonate (triflate, TfO). The leaving group may also be a halo (chlorine, bromine, iodine, or fluorine). In some embodiments, these compounds may be made from highly activated, dry K¹⁸F. Purification may be performed, e.g., via salt removal by reverse-phase chromatography (Sep-Pak).

In some embodiments, provided are methods of producing of a radiotracer compound of Formula I:

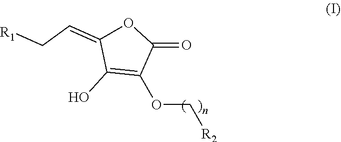

wherein:

R₁ is a substituted aryl or substituted heteroaryl, which is substituted with —X(CH₂)ₘ—R₃,

wherein X is O, NH, S or CH₂;

m is 0, 1, 2, 3, 4, 5, or 6;

R₃ is an ¹⁸F imaging moiety;

n is 0; and

R₂ is H;

or wherein:

R₁ is halo;

n is 0, 1, 2, 3, 4, 5, or 6; and

R₂ is a substituted aryl or substituted heteroaryl, which is substituted with —X(CH₂)ₘ—R₃,

wherein X is O, S, NH or CH₂;

m is 0, 1, 2, 3, 4, 5, or 6; and

R₃ is an ¹⁸F imaging moiety,

or a pharmaceutical acceptable salt thereof;
comprising:

(a) mixing a precursor compound of Formula (I);

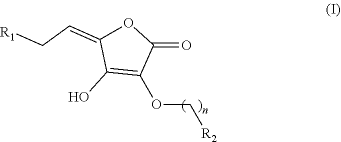

wherein R₁ is a substituted aryl or substituted heteroaryl, which is substituted with —X(CH₂)ₘ—R₃,
wherein X is O, NH, S or CH₂;

m is 0, 1, 2, 3, 4, 5, or 6;

R₃ is a leaving group;

n is 0; and

R₂ is H;

or wherein:

R₁ is halo;

n is 0, 1, 2, 3, 4, 5, or 6; and

R₂ is a substituted aryl or substituted heteroaryl, which is substituted with —X(CH₂)ₘ—R₃,

wherein X is O, S, NH or CH₂;

m is 0, 1, 2, 3, 4, 5, or 6;

R₃ is a leaving group selected from a sulfonate (e.g., tosylate) and halo,

with a solvent and a solution of [¹⁸F]F to obtain a reaction mixture;

(b) heating the reaction mixture obtained in step (a); and

(c) purifying the reaction mixture obtained in step (b), to produce the radiotracer compound.

Representative reaction solvents include, for example, dimethylformamide (DMF), N-methyl-2-pyrrolidone (NMP), dimethyl sulfoxide (DMSO), tetrahydrofuran (THF), ethyl acetate, dichloromethane, and chloroform. In some embodiments, the solvent is DMF. The reaction solution may be kept neutral or basic by the addition of a mild base (e.g., triethylamine or N,N-diisopropylethlamine (DIEA)).

Reactions may be carried out at ambient or elevated temperatures and may be protected from oxygen and water with a nitrogen atmosphere. For example, in some embodiments, the reaction is carried out at temperatures of from about 70° C. to about 150° C., from about 80° C. to about 120° C., or from about 90° C. to about 110° C. (or at least about 70° C., about 80° C., about 90° C., about 100° C., about 110° C., about 120° C., about 130° C., or at least about 140° C.). The reaction may be carried out for times ranging from about 1 to about 60 minutes, about 2 to about 50 minutes, about 3 to about 25 minutes, or about 5 to about 20 minutes.

Purification of the radiotracer may be performed using methods known in the art. For example, in some embodiments, the radiotracer is purified using reverse phase liquid chromatography (e.g., by employing a Sep-Pack).

In some embodiments, the radiolabeling precursor of compound of Formula (I) is:

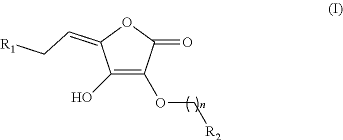

wherein:

R₁ is aryl substituted with —X(CH₂)ₘ—R₃, wherein X is O;

m is 0, 1, 2, 3, 4, 5, or 6;

R₃ is a sulfonate leaving group (e.g., tosylate);

n is 0; and

R₂ is H.

In some embodiments, the radiolabeling precursor compound of Formula (I) is:

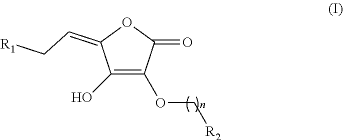

wherein:

R₁ is Cl;

n is 0, 1, 2, 3, 4, 5, or 6; and

R₂ is aryl substituted with —X(CH₂)ₘ—R₃, wherein X is O;

m is 0, 1, 2, 3, 4, 5, or 6; and

R₃ is a sulfonate leaving group (e.g., tosylate).

In some embodiments, the radiolabeling precursor of compound of Formula (I) is selected from:

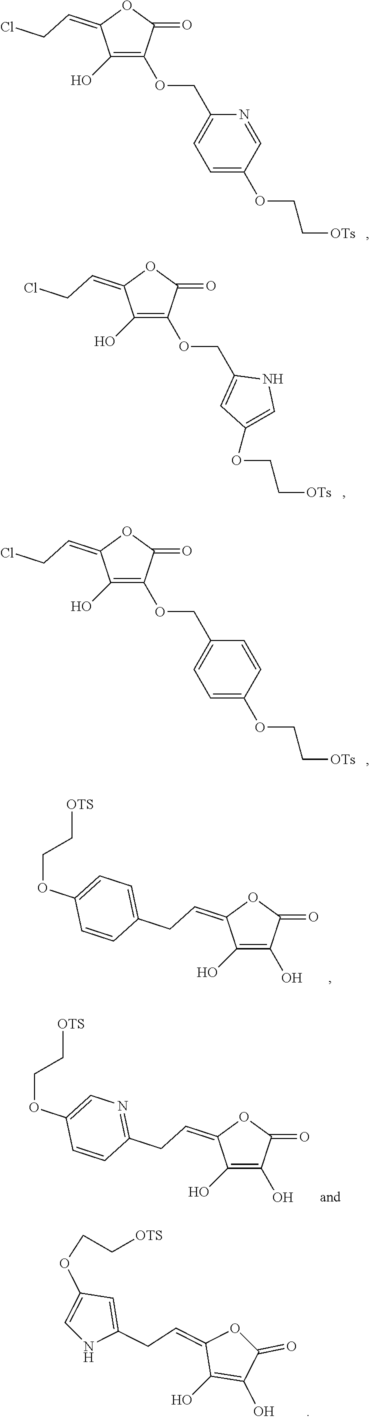

In some embodiments, the radiotracer is a compound selected from:

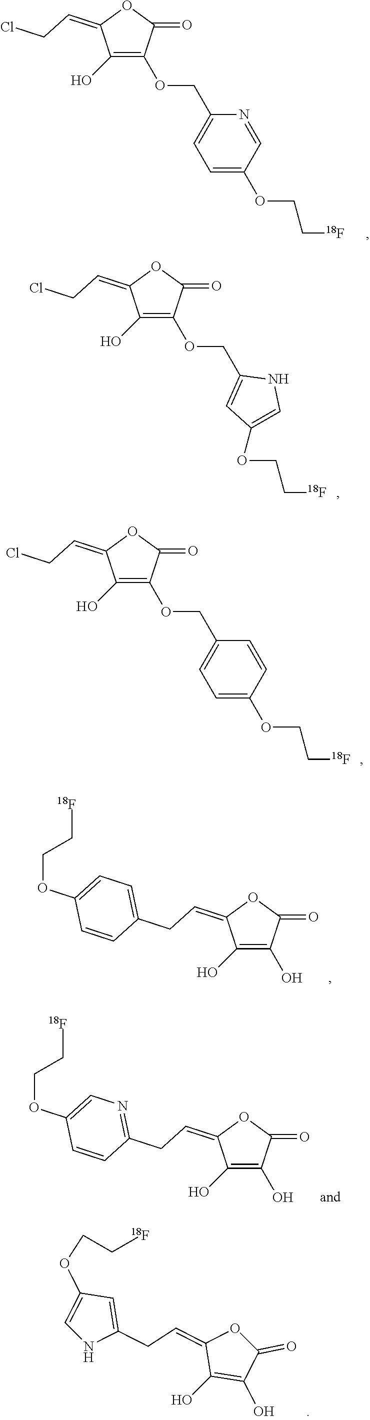 [SMILES: O=C1O/C(=C/CCl)C(O)=C1OCc1ccc(OCC[18F])cn1 ; O=C1O/C(=C/CCl)C(O)=C1OCc1cc(OCC[18F])c[nH]1 ; O=C1O/C(=C/CCl)C(O)=C1OCc1ccc(OCC[18F])cc1 ; O=C1O/C(=C/Cc2ccc(OCC[18F])cc2)C(O)=C1O ; O=C1O/C(=C/Cc2ccc(OCC[18F])cn2)C(O)=C1O ; O=C1O/C(=C/Cc2cc(OCC[18F])c[nH]2)C(O)=C1O]

In some embodiments, the method of the invention relates to the synthesis of a ¹¹C radiotracer comprising a compound of Formula I:

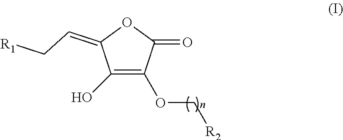

wherein:

R₁ is a substituted aryl or substituted heteroaryl, which is substituted with —X(CH₂)ₘ—R₃,

wherein X is O, NH, S or CH₂;

m is 0, 1, 2, 3, 4, 5, or 6;

R₃ is an ¹¹C imaging moiety;

n is 0; and

R₂ is H;

or wherein:

R₁ is halo;

n is 0, 1, 2, 3, 4, 5, or 6; and

R₂ is a substituted aryl or substituted heteroaryl, which is substituted with —X(CH₂)ₘ—R₃,

wherein X is O, S, NH or CH₂;

m is 0, 1, 2, 3, 4, 5, or 6;

R₃ is a ¹¹C imaging moiety,

or a pharmaceutically acceptable salt thereof.

In some embodiments, the ¹¹C imaging moiety is a ¹¹CH₃ imaging moiety. In general, such a radiotracer may be prepared by a Sₙ2 nucleophilic displacement reaction of ¹¹CH₃I with a nucleophile such as a hydroxyl group (i.e., a hydroxyl group attached to the ascorbate backbone).

In some embodiments, the radiolabeling precursor compound of Formula (I) is:

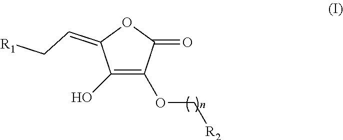

wherein:

R₁ is a substituted aryl or substituted heteroaryl, which is substituted with —X(CH₂)ₘ—R₃,

wherein X is O, NH, S or CH₂;

m is 0, 1, 2, 3, 4, 5, or 6;

R₃ is OH;

n is 0; and

R₂ is H;

or wherein:

R₁ is halo (e.g., Cl, Br, F, I);

n is 0, 1, 2, 3, 4, 5, or 6; and

R₂ is a substituted aryl or substituted heteroaryl, which is substituted with —X(CH₂)ₘ—R₃,

wherein X is O, S, NH or CH₂;

m is 0, 1, 2, 3, 4, 5, or 6;

R₃ is OH,

or a pharmaceutically acceptable salt thereof.

Additional methods and modifications to the above methods of synthesis may be apparent to the skilled person in view of the art. See, e.g., Tu et al., “C-11 Radiochemistry in Cancer Imaging Applications,” Curr Top Med Chem 2010, 10(11), 1060-1095; Dahl et al., “New methodologies for the preparation of carbon-11 labeled radiopharmaceuticals,” Clin Transl Imaging 2017, 5, 275-289; Solingapuram Sai et al., “Improved Automated Radiosynthesis of [¹¹C]PBR28,” Scientia Pharmaceutica 2015, 83(3), 413-427; Solingapuram Sai et al., “Automated radiochemical synthesis and biodistribution of [11C]l-α-acetylmethadol ([11C]LAAM),” Appl Radiat Isot 2014, 91, 135-140.

### III. Methods of Use for Imaging.

The active compounds taught herein are useful in methods of labeling cells, particularly cells having reactive oxygen species (ROS) and/or reactive nitrogen species (RNS). Such methods of labeling may include steps of contacting cells with an effective amount of a compound of the invention and then measuring gamma radiation emitted by the compound using any known methods in the art.

The levels of ROS and RNS in a cell can very. For example, a healthy cell typically has a lower ROS and/or RNS level compared to a diseased cell (e.g., a cancerous cell). In another example, the ROS and/or RNS level of two different types of diseased cells (e.g., two different cancerous cells) can vary, as well. The methods of the invention are able to differentiate between a healthy cell and a diseased cell or two different types of diseased cells based on the cellular uptake of the compounds of the invention due to the different levels of ROS and/or RNS present in each cell.

In some embodiments, cellular uptake of the compound of the invention into a diseased cell (e.g., a cancerous cell) is higher compared to the cellular uptake of the compound of the invention into a healthy cell. In some embodiments, the cellular uptake of the compound of the invention into a diseased cell is about 2-fold, about 3-fold, about 4-fold, about 5-fold, about 6-fold, about 7-fold, about 8-fold, about 9-fold, or about 10-fold higher compared to the cellular of the compound of the invention into a healthy cell.

In some embodiments, cellular uptake of the compound of the invention into a first diseased cell (e.g., a cancerous cell) is higher compared to the cellular uptake of the compound of the invention into a second diseased cell, wherein the first diseased cell is different compared to the second diseased cell. In some embodiments, the cellular uptake of the compound of the invention into a first diseased cell is about 2-fold, about 3-fold, about 4-fold, about 5-fold, about 6-fold, about 7-fold, about 8-fold, about 9-fold, or about 10-fold higher compared to the cellular of the compound of the invention into a second diseased cell.

Diseased and healthy cells contacted by the compound of the invention can be derived from any cell line and/or tissue. For example, in some embodiments, the cell is a from a cancer cell line. Example cancer cell lines include, but are not limited to, SCC-61, rSCC-61, G48, MD3-MD231, MCF-7, HepG2 (hepatocellular carcinoma), HeLa (cervix adenocarcinoma) and ACC1-activated fibroblasts, PC3, NSCLC (non-small cell lung cancer), U118 MG, HCC78, and 3T3 (cholangiocarcinoma). In some embodiments, the cells are from a diseased tissue such as, but not limited to, cancerous tissue, severely damaged neurodegenerative brain tissues, inflammatory tissues including colitis, chronic hepatitis, bacterial infections, cardiovascular/atherosclerotic plaque tissues. In general, the tissue is from a mammal, and in some embodiments the mammal is a human.

In some embodiments, the method of the invention measures the cellular uptake of the compound of the invention in vitro. In some embodiments, the method of the invention measures the amount of ROS and/or RNS in vitro. In some embodiments, the method of the invention measures the cellular uptake of the compound of the invention in vivo. In some embodiments, the method of the invention measures the amount of ROS and/or RNS in vivo.

In some embodiments, the compound of the invention exhibits an IC₅₀ values ranging from about 100 μM to about 300 μM, from about 150 μM to about 250 μM, or from about 180 μM to about 200 μM (or at least about 100 μM, about 110 μM, about 120 μM, about 130 μM, about 140 μM, about 150 μM, about 160 μM, about 170 μM, about 180 μM, about 190 μM, about 200 μM).

In some embodiments, the compound of the invention exhibits an IC₅₀ values ranging from about 500 μM to about 3 mM, or from about 750 μM to about 2 mM (or at least about 500 μM, about 600 μM, about 700 μM, about 750 μM, about 800 μM, about 900 μM, about 1 mM, about 1.25 mM, about 1.5 mM, about 1.75 mM, about 2 mM).

Another aspect of the invention relates to an active compound as taught herein useful in methods of labeling tissues in a subject, particularly tissues having reactive oxygen species (ROS) and/or reactive nitrogen species (RNS). Such methods of labeling may include steps of administering an active compound as taught herein to a subject, and then detecting gamma radiation emitted by the compound according to known methods in the art (e.g., by performing a PET scan on the subject), wherein detection of the gamma radiation indicates the presence ROS/RNS in the tissues.

In some embodiments, the tissues comprise cancerous tissues for cancer detection or treatment. Cancers may include, e.g., head and neck cancer, breast, colon, lung, prostate, brain or liver cancer. Other imaging applications include, but are not limited to, neuroimaging (e.g., Alzheimer's), diabetes, cardiology (e.g., cardiovascular diseases, atherosclerosis, hypertension, ischemia/reperfusion injury), and rheumatoid arthritis.

In some embodiments, the administering step is carried out by parenteral administration. For example, in some embodiments, the compound of the invention is administered by systemic injection and/or infusion. After administration, imaging the area of interest of the subject is carried out to locate any diseased tissue (e.g., a tumor).

The useful dosage to be administered and the particular mode of administration will vary depending upon such factors as age, weight, and particular region of the subject's body to be imaged, as well as the form of the pharmaceutical formulation. In some embodiments, the compounds of the invention may be included in formulations suitable for oral, rectal, topical, buccal (e.g., sub-lingual), parenteral (e.g., subcutaneous, intramuscular, intradermal, or intravenous) and transdermal administration. For example, formulations of the present invention suitable for parenteral administration conveniently comprise sterile aqueous preparations of the compound of the invention (e.g., radioactive compound), which preparations are preferably isotonic with the blood of the intended recipient. These preparations may be administered by means of subcutaneous, intravenous, intramuscular, or intradermal injection. Such preparations may conveniently be prepared by admixing the compound with water or a glycine buffer and rendering the resulting solution sterile and isotonic with the blood. Examples may also include pharmaceutical formulations comprising a suspension, emulsion, microsphere, liposomes or the like, as will be apparent to those skilled in the art.

In some embodiments, dosage may be administered at lower levels and increased levels until the desirable diagnostic and/or therapeutic effect is achieved. In some embodiments, the compound of the invention may be administered (e.g., by intravenous injection usually in saline solution) at a dose of about 0.1 to about 10-15 mCi per 50-70 kg body weight (and all combinations and subcombinations of dosage ranges and specific dosages therein). Imaging may be performed using techniques well known to the person of ordinary skill in the art.

A composition comprising compounds of the present disclosure may be administered at dosages ranging from about 0.5 μmol/kg to about 1.5 mmol/kg (and all combinations and subcombinations of dosage ranges and specific dosages therein).

Another aspect of the invention relates to a diagnostic kit for the preparation of the radioactive compounds of the invention for imaging ROS and/or RNS in a subject. Diagnostic kits of the present invention comprises one or more vials containing a predetermined amount of a compound of the present invention (e.g., a compound of the present invention containing a leaving group, e.g., a tosylate), and optionally one or more pharmaceutically acceptable excipients such as, but not limited to, buffers, stabilizing agents, and/or solvents. In some embodiments, the compound of the present invention is formulated as a pharmaceutically acceptable composition and/or pharmaceutically acceptable formulation, which is part of the diagnostic kit comprising one or more vials containing pharmaceutically acceptable excipients required for radiosynthesis to produce the radioactive compound of the invention.

The present invention is explained in greater detail in the following non-limiting Examples.

### EXAMPLES

### Example 1: Synthesis of (E)-5-(2-chloroethylidene)-3-(4-(2-fluoroethoxy)benzyloxy)-4-hydroxyfuran-2(5H)-one (KS1-OMe), (E)-5-(2-chloroethylidene)-3-(4-(2-methoxy)benzyloxy)-4-hydroxyfuran-2(5H)-one (KS1) and (E)-2-(4-(((5-(2-chloroethylidene)-4-hydroxy-2-oxo-2,5-dihydrofuran-3-yl)oxy)methyl)phenoxy)ethyl 4-methylbenzenesulfonate (KS1-OTs)

The synthetic scheme (see Scheme 1 above) was derived based on reported literature with slight modifications (Wittine et al., The new 5- or 6-azapyrimidine and cyanuric acid derivatives of 1-ascorbic acid bearing the free C-5 hydroxy or C-4 amino group at the ethylenic spacer: CD-spectral absolute configuration determination and biological activity evaluations. European Journal of Medicinal Chemistry 2011, 46 (7), 2770-2785; Wittine et al., Novel 1,2,4-triazole and imidazole derivatives of 1-ascorbic and imino-ascorbic acid: Synthesis, anti-HCV and antitumor activity evaluations. Bioorganic & Medicinal Chemistry 2012, 20 (11), 3675-3685; Gazivoda et al., The novel C-5 aryl, alkenyl, and alkynyl substituted uracil derivatives of 1-ascorbic acid: Synthesis, cytostatic, and antiviral activity evaluations. Bioorganic & Medicinal Chemistry 2007, 15 (2), 749-758; Raić-Malić et al., Novel Pyrimidine and Purine Derivatives of 1-Ascorbic Acid: Synthesis and Biological Evaluation. Journal of Medicinal Chemistry 1999, 42 (14), 2673-2678). Briefly, 5′, 6′ hydroxyl groups of L-ascorbic acid (1) was protected by acetone/acetyl chloride to obtain a ketal intermediate, 2. The ketal intermediate was then reacted with 4′-bromobenzyl substituted ethoxy alkanes, followed by acid deprotection to give 2′-benzyloxy, 5′,6′-dihydroxyl ascorbic acid, 3. Dehydration and selective 3′ debenzylation of the isolated product from the previous step 3 resulted in our desired analogs. Our chemical isolated yields of the derivatives KS1-OMe with p-methoxyphenyl, and KS1 with 2′-ethoxyfluoro and KS1-OTs with 2′-ethoxytosyl base were 38%, 52% and 48% respectively.

All intermediates and final compounds were completely characterized using ¹H-NMR and mass spectrometry. (E)-2-(4-(((5-(2-chloroethylidene)-4-hydroxy-2-oxo-2,5-dihydrofuran-3-yl)oxy)methyl)phenoxy)ethyl 4-methylbenzenesulfonate, KS1-OTs was obtained as a white solid, 38% yield and with ¹H NMR (400 MHz, CDCl₃): δ 10.15 (s, 1H), 7.82 (d, 2H, J=8.4 Hz), 7.38-7.33 (d, 2H, J=8.4 Hz), 7.27-7.22 (d, 2H, J=7.8 Hz), 6.79-6.77 (d, 2H, J=7.8 Hz), 5.46 (t, 1H, J=8.4 Hz), 5.12 (s, 2H), 4.39-4.36 (m, 2H), 4.29 (d, 2H, J=8.4 Hz), 4.18-4.16 (m, 2H), 2.46 (s, 3H); MS: 481.96 [M+H]⁺. The nonradioactive standard, (E)-5-(2-chloroethylidene)-3-(4-(2-fluoroethoxy)benzyloxy)-4-hydroxyfuran-2(5H)-one, KS1 was isolated as a light brown solid, 24% yield and ¹H NMR (300 MHz, CDCl₃): δ 10.12 (s, 1H), 7.32-7.31 (d, 2H, J=2.8 Hz), 6.89-6.88 (d, 2H, J=2.8 Hz), 5.50-5.46 (t, 1H, J=7.1 Hz), 5.19 (s, 2H), 4.86-4.83 (m, 1H), 4.70-4.65 (m, 1H), 4.39-4.37 (d, 2H, J=10.4 Hz), 4.28-4.27 (m, 1H), 4.19-4.16 (m, 1H); MS: 329.05 [M+H]⁺. KS1-OMe: ¹H NMR (400 MHz, CDCl₃): δ 11.11 (s, 1H), 9.37 (s, 1H), 7.13-7.11 (d, J=8.4 Hz, 2H), 6.87-6.85 (d, J=8.4 Hz, 2H), 5.47-5.42 (m, 1H), 3.71 (s, 3H), 3.49-3.46 (dd, J=3.6 Hz, 7.6 Hz, 2H). ES-MS (m/z): 248.23 (M+1).

### Example 2: Synthesis of KS2-OMe, KS2, and KS2-OTs

6′ aryl substituted L-ascorbic acid KS2-OMe and KS2 were prepared as depicted in Scheme 2 above (see references cited in Example 1 above; Gazivoda et al., Synthesis, structural studies, and cytostatic evaluation of 5,6-di-O-modified 1-ascorbic acid derivatives. Carbohydrate Research 2006, 341 (4), 433-442; Cheng et al., Advances in chitosan-based superabsorbent hydrogels. RSC Advances 2017, 7 (67), 42036-42046; Gautam et al., Stereoselective synthesis of (+)-(1R,2S,5S,7R)-2-hydroxy-exo-brevicomin. Tetrahedron Letters 2010, 51 (32), 4199-4201; Schachtner et al., Organoalane-mediated isomerization of ascorbic and isoascorbic acid derivatives. Tetrahedron: Asymmetry 1996, 7 (11), 3263-3276). Briefly, L ascorbic acid (1) underwent epoxidation and benzylation to form a di-O-benzyl substituted epoxide intermediate, 3,4-bis(benzyloxy)-5-(oxiran-2-yl)furan-2(5H)-one, 2B. When subjected to ring opening reaction with 4-aryl iodide, this epoxide produced an aryl substituted furanose derivative, 3B. After dehydration and debenzylation reactions the results were the final compounds, KS2-OMe (with p-methoxyphenyl), and KS2 (with 2′ethoxyfluoro base) in 27% and 31% isolated chemical yields respectively. Simultaneously, the corresponding [¹⁸F]-tosyl precursor KS2-OTs was also synthesized by similar methods (38% chemical yield).

All intermediates and final compounds were fully characterized using ¹H-NMR and high resolution mass spectrometry. KS2: ¹H NMR (400 MHz, DMSO-d6): δ 11.05 (s, 1H), 9.36 (s, 1H), 7.14-7.12 (d, J=8.4 Hz, 2H), 6.91-6.89 (d, J=8.4 Hz, 2H), 5.46-5.42 (t, J=8 Hz, 1H), 4.78-4.64 (m, 2H), 4.23-4.14 (s, 2H), 3.42-3.47. (d, J=8 Hz, 2H); ES-MS (m/z): 280.25 (M+1). KS2-OTs: ¹H NMR (400 MHz, DMSO-d6): δ 11.04 (s, 1H), 9.42 (s, 1H), 7.79-7.77 (d, J=8 Hz, 2H), 7.427-7.407 (d, J=8 Hz, 2H) 7.14-7.12 (d, J=8.4 Hz, 2H), 6.91-6.89 (d, J=8.4 Hz, 2H), 5.36-5.32 (t, J=8 Hz, 1H), 4.35-4.31 (m, 2H), 4.19-4.11 (s, 2H), 3.42-3.47. (d, J=8 Hz, 2H), 2.36 (s, 3H); ES-MS (m/z): 433.1 (M+1)

### Example 3: Production of PET Radiotracer [¹⁸F]KS1 & [¹⁸F]KS2

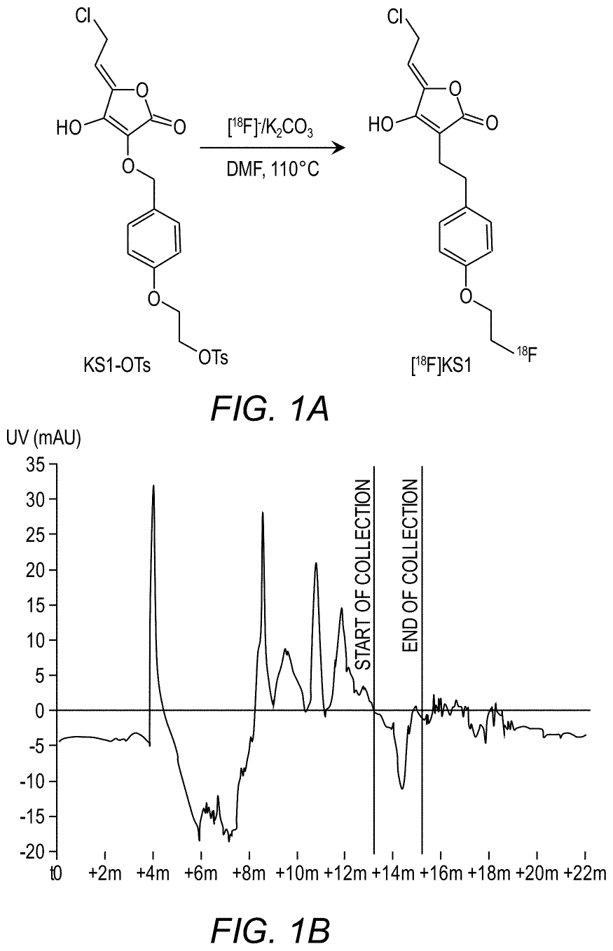

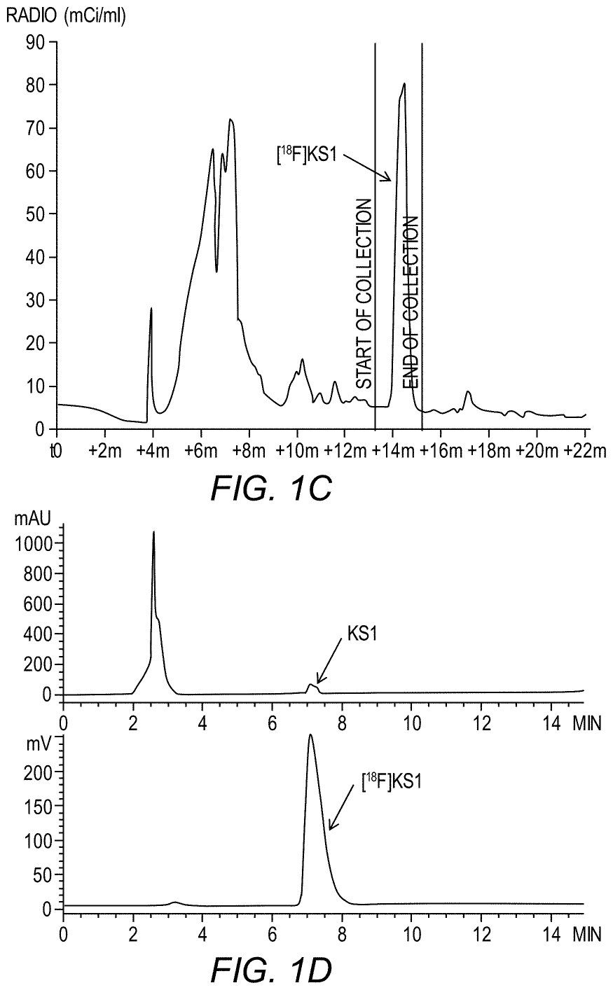

With both the precursor tosylates and non-radioactive F-19 reference standard in hand, the radiochemical synthesis of [¹⁸F]KS1 and [¹⁸F]KS2 were optimized on the TRASIS AIO radiochemistry module (FIG. 1A-1D) (Li et al., Automation of the Radiosynthesis of Six Different 18F-labeled radiotracers on the AllinOne. EJNMMI Radiopharmacy and Chemistry. 2017; 1:15), following the typical [¹⁸F]-based nucleophilic substitution reaction from the corresponding ascorbate tosylate, as descried in Scheme 2 (Sai et al., Radiolabeling and initial biological evaluation of [¹⁸F]KBM-1 for imaging RAR-α receptors in neuroblastoma. Bioorganic & medicinal chemistry letters. 2017; 27:1425-7). Briefly, [¹⁸F]F⁻ produced from our GE PETtrace cyclotron was azeotropically dried and reacted with corresponding tosylate precursor in DMF at 110° C. for 15 min. Semi-preparative HPLC separation and solid phase C18 sepPak purification, and elution with 10% absolute ethanol in saline resulted in [¹⁸F]KS1 (Solingapuram Sai et al., 18F-AFETP, 18F-FET, and 18F-FDG Imaging of Mouse DBT Gliomas. Journal of Nuclear Medicine. 2013; 54:1120-6; Solingapuram Sai et al., Radiosynthesis and Evaluation of [11C]HD-800, a High Affinity Brain Penetrant PET Tracer for Imaging Microtubules. ACS Medicinal Chemistry Letters. 2018; 9:452-6). The isolated radioactive product was used for quality control analyses, in vitro cell uptake, and animal studies. The chemical and radiochemical purity and specific activity of the collected radioactive aliquots were determined by HPLC injection on a QC C18 reverse phase column.

Radiochemical synthesis, including [¹⁸F]F⁻ transfer, reaction, HPLC purification, and radiotracer formulation, was completed within 65 min (FIG. 1C). Injection of [¹⁸F]KS1 showed a single radioactive peak with minimal UV absorbance, indicating good specific activity (FIG. 1D). The radioactive peaks were further authenticated by performing a co-injection with their corresponding non-radioactive standard KS1 and KS2, which displayed similar retention times. [¹⁸F]KS1 and [¹⁸F]KS2 were synthesized with high radiochemical purity (>94%) and high specific activity ˜2760-3820 mCi/μmol (decay corrected to end of synthesis; EOS). [¹⁸F]KS1 and [¹⁸F]KS1 were produced in 28% and 30% decay corrected radiochemical yield (n>10), respectively.

### Example 4: In Vitro Testing of KS1 and KS2 with Ascorbate as a Reference Standard at Lower Concentrations

A panel of two cell lines derived from patients with head and neck squamous cell carcinoma (HNSCC) SCC-61 and rSCC-61 were tested in a genetically matched model of radiation resistance using systems-level analyses and complementary assays (Xiaofei et al., Modulators of Redox Metabolism in Head and Neck Cancer. Antioxidants & Redox Signaling; Chen et al., Analysis of DNA methylation and gene expression in radiation-resistant head and neck tumors. Epigenetics 2015, 10 (6), 545-561; Mims et al., Energy Metabolism in a Matched Model of Radiation Resistance for Head and Neck Squamous Cell Cancer. Radiation research 2015, 183 (3), 291-304; Reisz et al., Effects of Ionizing Radiation on Biological Molecules—Mechanisms of Damage and Emerging Methods of Detection. Antioxidants & Redox Signaling 2014, 21 (2), 260-292; Poole et al., Fluorescent and Affinity-Based Tools To Detect Cysteine Sulfenic Acid Formation in Proteins. Bioconjugate Chemistry 2007, 18 (6), 2004-2017). SCC-61 and rSCC-61 cells showed differences in: (a) response to radiation (SCC-61: D0 1.3, rSCC-61 D0 2.0); (b) response to the EGFR targeted inhibitor erlotinib (SCC-61: IC50>50 μM; rSCC-61: IC50 4.5 μM); and, (c) cellular phenotype: SCC-61 is mesenchymal while rSCC-61 is epithelial. Quantitative proteomics revealed: 1) upregulation of antioxidant proteins (Prx1-3, GST-pi) and downregulation of MnSOD in rSCC-61; cumulatively these results explain the differences in intracellular ROS in rSCC-61 (low) and SCC-61 (high); 2) upregulation of DNA replication and base excision repair in rSCC-61 correlating with less DNA damage in rSCC-61; 3) downregulation of ECM-receptor interaction and focal adhesion in rSCC-61; and, 4) upregulation of keratins, fatty acid synthase, and Cl metabolism.

As a first-line tool to screen KS1 and KS2: SCC-61 has higher intracellular ROS than rSCC-61. MitoSOX is a mitochondrion-targeted dihydroethidium based derivative that primarily detects ROS (superoxide anion) produced within mitochondria. Mitosox assays without any ligands were used as controls. The two cell lines were treated with KS1, KS2 and ascorbate (10.0 μM) and incubated for an hour. MitoSOX (1.0 μM) in phenol red-free DMEM/F12 media was added and incubated for an additional 10 min. The cells were then washed with PBS and imaged in the same media.

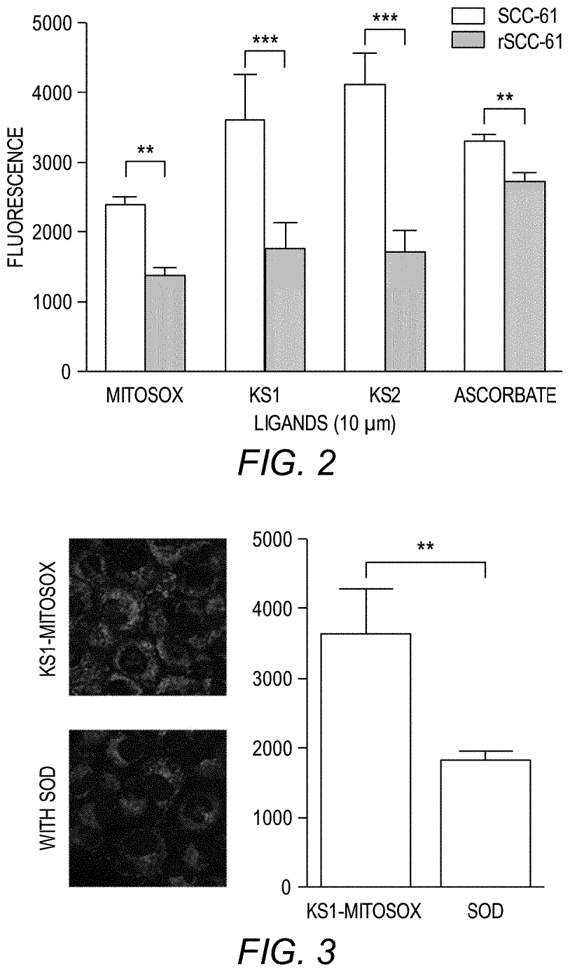

The two ascorbate ligands KS1 and KS2 exhibited a similar fluorescence as ascorbate. All three compounds demonstrated higher fluorescence in SCC-61 cell line over rSCC-61 cells (FIG. 2). Good correlations were found between increasing concentrations of the substrates (KS1, KS2 and ascorbate) from 1.0 μM to 100 μM and increased fluorescence. KS1 and KS2 exhibited ˜10 fold higher selectivity between the two cell lines compared to ascorbate selectivity, i.e., a significant difference in the uptake of our tracers compared to ascorbate.

### Example 5: Fluorescence MitoSOX Assay of KS1

A panel of SCC-61 and rSCC-61 cell lines derived from patients with HNSCC, a genetically matched model of radiation resistance developed previously (see references cited above in Example 4). The molecular and cellular properties of this model have been well characterized using systems-level analyses and complementary assays SCC-61 and rSCC-61 cells show differences in: (a) response to radiation (SCC-61: D0 1.3, rSCC-61 D0 2.0); (b) response to the EGFR targeted inhibitor erlotinib (SCC-61: IC₅₀>50 μM; rSCC-61: IC₅₀ 4.5 μM); (c) cellular phenotype: SCC-61 is mesenchymal while rSCC-61 is epithelial; and importantly (d) ROS levels: SCC-61 has higher intracellular ROS than rSCC-61 (Mims et al., Energy Metabolism in a Matched Model of Radiation Resistance for Head and Neck Squamous Cell Cancer. Radiation research. 2015; 183:291-304). MitoSOX is a mitochondrion-targeted dihydroethidium based derivative that primarily detects ROS, especially superoxide anion radical, produced within mitochondria (Kuznetsov et al., Mitochondrial ROS production under cellular stress: comparison of different detection methods. Analytical and Bioanalytical Chemistry. 2011; 400:2383-90).

In addition to testing KS1 with the MitoSOX assay, we compared our results with no ligand controls and ascorbate as a reference standard. The two cell lines were treated with KS1 and ascorbate (10.0 μM) and incubated for an hour. MitoSOX (1.0 μM) in phenol red-free DMEM/F12 media was added and incubated for an additional 10 min. The cells were then washed with PBS and imaged in the same media. Ligand specificity is commonly determined by pretreating the cells with high concentrations of blockers. Superoxide dismutase (SOD), an enzyme that selectively suppresses accumulation of ROS-superoxide peroxide anions, was added as blocker (1.0 mM) 60 min before the treatment of KS1 (Bielski et al., Chemistry of ascorbic acid radicals. in Ascorbic Acid: Chemistry, Metabolism, and Uses. Chapter. 1982; 4:81-100). Fluorescence was subsequently measured and its intensity was quantified in fu (fluorescence units).

KS1 and ascorbate showed a similar amount of fluorescence (FIG. 2). Both KS1 and ascorbate demonstrated higher fluorescence in the higher ROS SCC-61 cells compared to the lower ROS rSCC-61 cells. Good correlations were found between increasing concentrations of KS1 (and ascorbate) from 1.0 μM to 100 μM and increased fluorescence. KS1 exhibited ˜2.2-fold higher differential selectivity between the high- and low-ROS cell lines compared to ascorbate (FIG. 2). In order to further establish specificity of KS1 for ROS, the same MitoSOX assay was performed by pretreating the SCC-61 cells with SOD (1.0 mM), an ROS blocker, for 60 min. Fluorescent uptake decreased by ˜50%, demonstrating KS1 specificity (data not shown).

### Example 6: Determination of Specificity of the Ligand

Ligand specificity is commonly determined by pretreating the cells with high concentrations of blockers. We used superoxide dismutase (SOD), an enzyme that selectively suppresses accumulation of ROS-superoxide peroxide anions (Bielski et al., Chemistry of ascorbic acid radicals. in Ascorbic Acid: Chemistry, Metabolism, and Uses. Chapter 1982, 4, 81-100). The same MitoSOX assay with KS1 and KS2 (10 μM) was performed by pretreating the SCC-61 cells with SOD (1.0 mM) for 60 min. As seen in FIG. 3, fluorescent intensity of KS1-mitoSOX was 3631, and with SOD it was 1842. Uptake decreased by ˜50% (KS1) and ˜30% (KS2), demonstrating specificity of the ligands. SOD specificity suggests that these agents could be superoxide peroxide (ROS) targeting agents (Chu et al., Development of a PET Radiotracer for Noninvasive Imaging of the Reactive Oxygen Species, Superoxide, in vivo. Organic & biomolecular chemistry 2014, 12 (25), 4421-443). Initial preliminary in vitro ROS assay demonstrated a) similar mode of action to that of ascorbate (at lower concentrations) and b) high ROS binding potency and specificity of KS1 and KS2.

### Example 7: Cytotoxicity of KS1 and KS2 in Tumor Cells with Ascorbate as a Reference at Higher Concentrations

There is growing evidence for a therapeutic role of ascorbate against several cancer lines (Creagan et al., Failure of High-Dose Vitamin C (Ascorbic Acid) Therapy to Benefit Patients with Advanced Cancer. New England Journal of Medicine 1979, 301 (13), 687-690; Chen et al., Pharmacologic ascorbic acid concentrations selectively kill cancer cells: Action as a pro-drug to deliver hydrogen peroxide to tissues. Proceedings of the National Academy of Sciences of the U.S. Pat. No. 2,005,102 (38), 13604-13609; Prasad et al., High Doses of Multiple Antioxidant Vitamins: Essential Ingredients in Improving the Efficacy of Standard Cancer Therapy. Journal of the American College of Nutrition 1999, 18 (1), 13-25). Recent clinical trial data suggests that high-dose ascorbate (oral and/or intravenous) could increase the effectiveness of cancer chemotherapy in many solid cancer therapies (López-Lázaro, Dual role of hydrogen peroxide in cancer: Possible relevance to cancer chemoprevention and therapy. Cancer Letters 2007, 252 (1), 1-8; Li et al., New Developments and Novel Therapeutic Perspectives for Vitamin C. The Journal of Nutrition 2007, 137 (10), 2171-2184; Klimant et al., Intravenous vitamin C in the supportive care of cancer patients: a review and rational approach. Current Oncology 2018, 25 (2), 139-148). The primary objective of this experiment was to determine and understand the cytotoxic effect of the new agents at relative higher concentrations, compared to ascorbate. Cancer cell lines were selected based on large literature evidence of oxidative stress management in clinical settings of cancer treatment. Determination of half-maximal (50%) inhibitory concentration (IC₅₀) is essential for understanding the pharmacological and biological characteristics of any active agent (Griffiths et al., Drug Design and Testing: Profiling of Antiproliferative Agents for Cancer Therapy Using a Cell-Based Methyl-[3H]-Thymidine Incorporation Assay. In Cancer Cell Culture: Methods and Protocols, Cree, I. A., Ed. Humana Press: Totowa, N.J., 2011; pp 451-465).

We obtained IC₅₀ values of KS1, KS2 and ascorbate in patient-derived cancer cell lines of (a) HNSCC (SCC-61), (b) glioblastoma (G48), (c) breast cancer (MD3-MB-231), and (d) PCa (PC3) using the MTT assay (96-well plate assay) (He et al., The changing 50% inhibitory concentration (IC(50)) of cisplatin: a pilot study on the artifacts of the MTT assay and the precise measurement of density-dependent chemoresistance in ovarian cancer. Oncotarget 2016, 7 (43), 70803-70821; Sobottka et al., Assessment of antineoplastic agents by MTT assay: partial underestimation of antiproliferative properties. Cancer chemotherapy and pharmacology 1992, 30 (5), 385-393). The cells were seeded at density 5×10³ cells/well. After 24 h pre-incubation period, cells were treated with 0.01 mM to 2.0 mM of freshly prepared KS1 and KS2 along with ascorbate (n=3) for 48 h. 0.2% of DMSO served as vehicle control. After 48 h, the solutions were removed from all plates and MTT assays performed following the published protocols. IC₅₀ values are presented as mean±SD from at least three independent experiments.

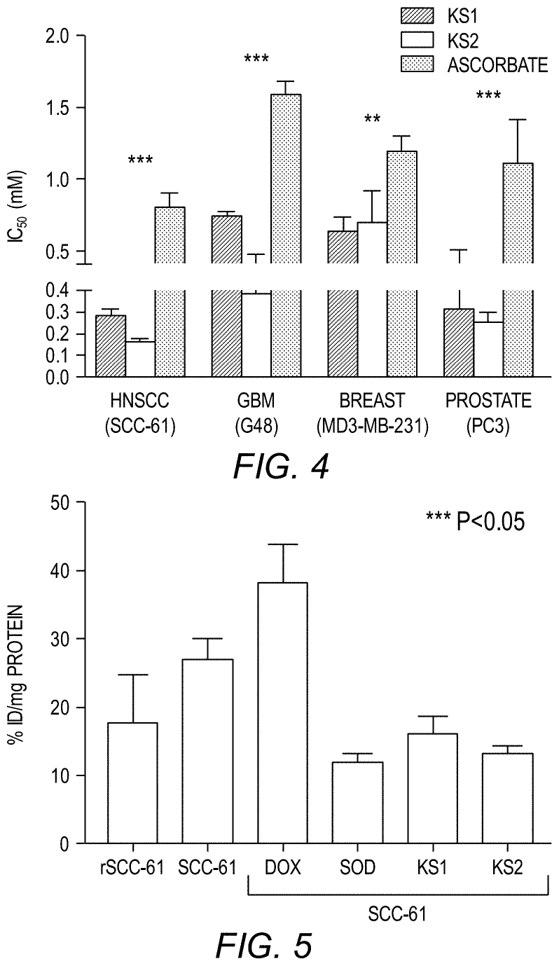

IC₅₀ of KS1 and KS2 in SCC-61 cell line (HNSCC) was 0.288 mM and 0.166 mM, respectively, while ascorbate was 0.815 mM. In PC3 cell line, KS1 and KS2 displayed IC₅₀ of 0.311 mM and 0.251 mM respectively while ascorbate's was 1.12 mM. Furthermore, in our preliminary data of Specific Aim 3A/3B (blocking studies), we injected 15 mg/kg of our analog KS1 and did not observe any toxicity in normal organs. Importantly, the IC₅₀ values of KS1 and KS2 were in the range of reported potent ascorbate derivatives, IC₅₀ values of 0.181-0.2 mM (Gazivoda et al., Synthesis, structural studies, and cytostatic evaluation of 5, 6-di-O-modified L-ascorbic acid derivatives. Carbohydrate research 2006, 341 (4), 433-442). FIG. 4 shows high potency of KS1 and KS2 over ascorbate in all four cancer cell lines, with >5 fold selectivity in SCC-61 and PC3 lines.

### Example 8: Cell Uptake Studies of [¹⁸F]KS1

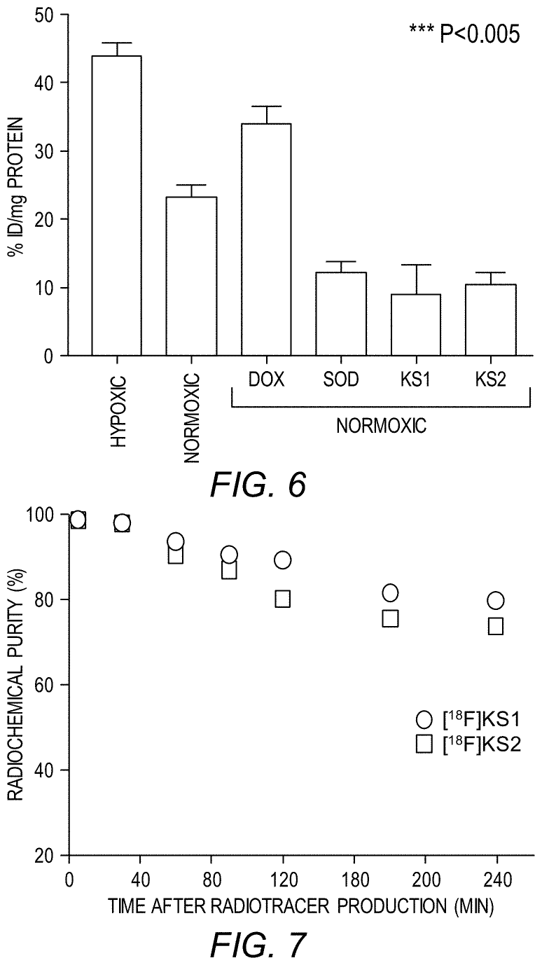

ROS efficacy was evaluated in two tumor cell lines: (1) SCC-61 and rSCC-61 cells (HNSCC) (FIG. 5), and (2) PC3 (PCa) cell lines under normoxic and hypoxic conditions (FIG. 6) following previously published protocols by our group (Sai et al., Peptide-based PET imaging of the tumor restricted IL13RA2 biomarker. Oncotarget 2017, 5; Yamamoto et al., Correlation of 18F-FLT and 18F-FDG uptake on PET with Ki-67 immunohistochemistry in non-small cell lung cancer. European Journal of Nuclear Medicine and Molecular Imaging 2007, 34 (10), 1610-1616; Solingapuram Sai et al., Radiolabeling and initial biological evaluation of [18F]KBM-1 for imaging RAR-α receptors in neuroblastoma. Bioorganic & Medicinal Chemistry Letters 2017, 27 (6), 1425-1427). Several ROS blockers including fresh solution of non-radioactive compounds KS1, KS2, SOD (10 μM) in the cell media were added to the seeded tumor cells 1.0 h prior to radiotracer addition (n=6 per blocker). All the cells were then treated with [¹⁸F]KS1 (2.0 μCi/well, UV mass=0.4 μg/mL and S.A=3100 mCi/μmol) and incubated for 60 min (n=6) at 37° C. All the cells (both with and without blockers) were washed and lysed with lysate buffer solution. Lysate samples from each well were collected for gamma counting.

Similarly, in another experiment, human PCa-PC3 cells were cultured under normoxic (˜21% O₂) or hypoxic (1% O₂) conditions for 48 h. Thereafter, similar to HNSCC cells (detailed above) all assay steps including blocker treatment, radiotracer addition, incubations, washings and lysis were carried out under normoxic or hypoxic conditions. All hypoxia experiments were performed in a hypoxia chamber (Biospherix X3 Xvivo system). Plates without radioactivity were used as controls. Additional aliquots were taken from each well to measure protein concentration. The counts per minute values of each well were normalized to the amount of radioactivity added to each well and was expressed as percent uptake relative to the control condition. The data were expressed as % ID/mg of protein present in each well.

Radioactive uptake in SCC-61 cells was ˜1.5-fold higher compared to rSCC-61 uptake (FIG. 5). Importantly, the uptake was successfully blocked by SOD (˜60%), and by nonradioactive KS1 (˜40%). Additionally, treatment with the ROS inducer, doxorubicin increased the [¹⁸F]KS1 uptake by ˜30%.

To further test the specificity of [¹⁸F]KS1 for ROS, radioactive uptake in PCa cells was tested under hypoxic conditions, as several studies have shown that hypoxic conditions promote ROS generation and oxidative stress (Kumar et al., Oxidative Stress Is Inherent in Prostate Cancer Cells and Is Required for Aggressive Phenotype. Cancer Research. 2008; 68:1777-85; Pinthus et al., Androgen Induces Adaptation to Oxidative Stress in Prostate Cancer: Implications for Treatment with Radiation Therapy. Neoplasia (New York, N.Y.). 2007; 9:68-80; Cervellati et al., Hypoxia induces cell damage via oxidative stress in retinal epithelial cells. Free Radical Research. 2014; 48:303-12). A reported hypoxia model of PCa cells was tested in which the uptake of [¹⁸F]KS1 was evaluated in PC3 cells cultured under hypoxic conditions versus normoxic conditions.

Radioactive uptake of [¹⁸F]KS1 in PC3 under hypoxic conditions was ˜2-fold higher than the normoxic conditions (FIG. 6), further indicating the ability of [¹⁸F]KS1 to measure ROS (Schlaepfer et al., Hypoxia induces triglycerides accumulation in prostate cancer cells and extracellular vesicles supporting growth and invasiveness following reoxygenation. Oncotarget. 2015; 6:22836-56; Liu et al., Hypoxia Accelerates Aggressiveness of Hepatocellular Carcinoma Cells Involving Oxidative Stress, Epithelial-Mesenchymal Transition and Non-Canonical Hedgehog Signaling. Cellular Physiology and Biochemistry. 2017; 44:1856-68). Importantly, this uptake was specific and due to ROS because it was blocked by SOD (˜48%), and nonradioactive KS1 (˜61%). Furthermore, doxorubicin increased the radioactive uptake by ˜15%, again confirming ROS specificity.

These promising in vitro cell uptake data in different cancer cell lines and through manipulations of ROS levels in vitro demonstrate excellent binding and specificity of [¹⁸F]KS1 towards ROS levels in tumor cells.

### Example 9: Serum Stability of [¹⁸F]KS1 and [¹⁸F]KS2

The ex vivo serum stability of [¹⁸F]KS1 and [¹⁸F]KS2 were analyzed in human serum samples, following previously published methods (Sai et al., Peptide-based PET imaging of the tumor restricted IL13RA2 biomarker. Oncotarget 2017, 5; Sattiraju et al., IL13RA2 targeted alpha particle therapy against glioblastoma. Journal of Nuclear Medicine 2016, 57 (supplement 2), 634; Wang et al., Evaluation of F-18-labeled amino acid derivatives and [18F]FDG as PET probes in a brain tumor-bearing animal model. Nuclear Medicine and Biology 2005, 32 (4), 367-375). Briefly, radiotracer was added to the human serum sample, and incubated at 37° C. Radioactive serum mixture was injected into the QC-HPLC system at 5 min, 30 min, 1 h, 1.5 h, 2 h, 2.5 h, and 3 h post radiotracer synthesis. Radiolysis through defluorination and/or oxidation of the radiotracers (if any) can be seen as new radiochemical peaks at different retention times (Rₜ) than the original product peak (Jin et al., A promising F-18 labeled PET radiotracer (−)-[18F]VAT for assessing the VAChT in vivo. Journal of Nuclear Medicine 2015, 56 (supplement 3); Koglin et al., [F-18]BAY 85-8050: A novel tumor specific probe for PET imaging—Preclinical results. Journal of Nuclear Medicine 2010, 51 (supplement 2), 1535; Pacelli et al., Imaging COX-2 expression in cancer using PET/SPECT radioligands: current status and future directions. Journal of Labelled Compounds and Radiopharmaceuticals 2014, 57 (4), 317-322; Harada et al., Characterization of the radiolabeled metabolite of tau PET tracer 18F-THK5351. European Journal of Nuclear Medicine and Molecular Imaging 2016, 43 (12), 2211-2218).

[¹⁸F]KS1 was ˜90% and [¹⁸F]KS2 was ˜80% intact in serum after 120 min of tracer synthesis (FIG. 7). Post 2 h, new radioactive peaks (just before retention times of product peaks) were seen (data not shown). Mass spec analysis are being carried out to investigate any possible reactions of the radiotracers. Overall, both the radiotracers showed ˜80% serum stability ex vivo and demonstrated minimal radiolysis through defluorination and/or oxidation of the radiotracers by relative lack of new radiochemical peaks at different retention times (Rₜ) compared to the original product peak, and are hence suitable for further in vivo studies (Jin et al., A promising F-18 labeled PET radiotracer (−)-[¹⁸F]VAT for assessing the VAChT in vivo. Journal of Nuclear Medicine. 2015; 56:4; Koglin et al., [F-18]BAY 85-8050: A novel tumor specific probe for PET imaging—Preclinical results. Journal of Nuclear Medicine. 2010; 51:1535. Pacelli et al., Imaging COX-2 expression in cancer using PET/SPECT radioligands: current status and future directions. Journal of Labelled Compounds and Radiopharmaceuticals. 2014; 57:317-22; Harada et al., Characterization of the radiolabeled metabolite of tau PET tracer 18F-THK5351. European Journal of Nuclear Medicine and Molecular Imaging. 2016; 43:2211-8).

[¹⁸F]KS1 is used for the following studies due to (a) slightly higher stability, (b) a relatively better in vitro ROS profile over KS2, and (c) lower costs associated with tracer productions.

### Example 10: In Vivo Evaluations of [¹⁸F]KS1

Athymic nude mice (Taconic Farms) were housed in a pathogen-free facility of the Animal Research Program at Wake Forest School of Medicine under a 12:12-h light/dark cycle and fed ad libitum. All animal experiments were conducted under IACUC approved protocols in compliance with the guidelines for the care and use of research animals established by Wake Forest Medical School Animal Studies Committee. PC3 cells (1×10⁵ cells suspended in 10 μL matrigel) were implanted in the left flank of nude mice (25-30 g) as described previously [61-63]. Mice bearing subcutaneous human PCa PC3 tumors were separated into two groups for baseline and blockade studies (n=3/group) and underwent microPET imaging under ˜1% isoflurane-oxygen anesthesia (Reuter et al., Oxidative stress, inflammation, and cancer: How are they linked? Free radical biology & medicine. 2010; 49:1603-16. doi:10.1016/j.freeradbiomed; Sosa et al., Oxidative stress and cancer: An overview. Ageing Research Reviews. 2013; 12:376-90; Singh et al., Silibinin suppresses in vivo growth of human prostate carcinoma PC-3 tumor xenograft. Carcinogenesis. 2007; 28:2567-74). Mice were intravenously injected with ˜100±10 μCi of [¹⁸F]KS1 and 45 min later were scanned for 20 min using a TriFoil microPET scanner. KS1 (15 mg/kg) was used as a blocking agent and was injected 45 min before the radiotracer injection. Standard biodistribution studies were conducted in mice bearing PCa tumors to confirm in vivo binding of [¹⁸F]KS1. Mice were intravenously injected with [¹⁸F]KS1 (˜100 and euthanized after 30 min and 60 min of tracer injections (3 mice/time point). Samples of tumor, blood, brain, heart, lung, liver, spleen, pancreas, kidney, muscle and bone were harvested, weighed, and gamma counted with a standard dilution of the injectate (Chu et al., Development of a PET Radiotracer for Noninvasive Imaging of the Reactive Oxygen Species, Superoxide, in vivo. Organic & biomolecular chemistry. 2014; 12:4421-31; Sai et al., Radiolabeling and initial biological evaluation of [(18)F]KBM-1 for imaging RAR-α receptors in neuroblastoma. Bioorganic & medicinal chemistry letters. 2017; 27:1425-7; Kumar et al., Radiosynthesis and in Vivo Evaluation of [11C]MPC-6827, the First Brain Penetrant Microtubule PET Ligand. Journal of Medicinal Chemistry. 2018; 61:2118-23). The percentage of the injected dose per gram of tissue (% ID/g) was calculated.

### Results:

MicroPET Imaging Studies

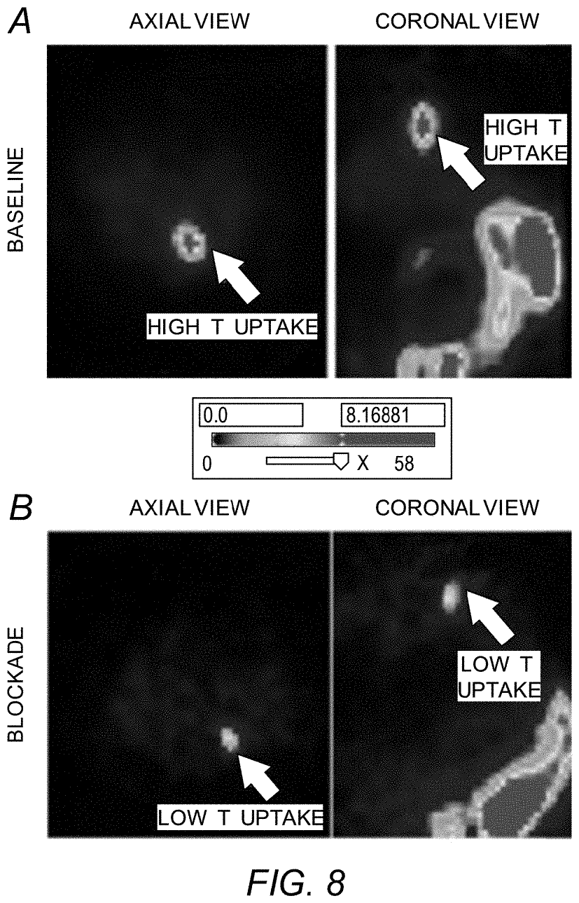

To evaluate the first in vivo imaging characteristics of [¹⁸F]KS1 in a tumor model, microPET imaging studies were performed. Using basic region of interest (ROI) analysis on the mPET scans, [¹⁸F]KS1 demonstrated (a) high tumor uptake and (b) successful blocking of uptake with nonradioactive KS1 pretreatment (˜3-fold lower than baseline), signifying retained high selectivity and specificity of [¹⁸F]KS1 in vivo (FIG. 8).

In Vivo Biodistribution:

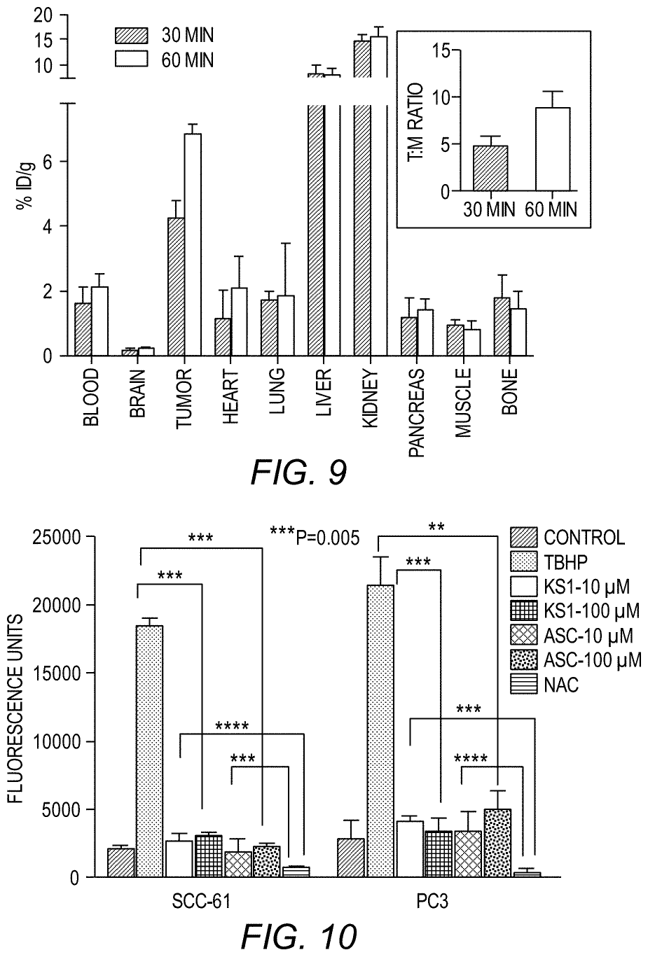

Results of standard biodistribution studies in mice implanted with PCa tumors (n=3/time point) were obtained (FIG. 9). From 30 min to 60 min post injection, [¹⁸F]KS1 displayed ˜1.7-fold increased tumor uptake: % ID/g of 4.23±0.531 (30 min) to 6.81±0.33 (60 min). Bone uptake was lowered from % ID/g of 1.741±0.71 (30 min) to 1.381±0.561 (60 min), suggesting no significant metabolic defluorination in vivo (Sai et al., Radiolabeling and initial biological evaluation of [(18)F]KBM-1 for imaging RAR-α receptors in neuroblastoma. Bioorganic & medicinal chemistry letters. 2017; 27:1425-7). Additionally, there was >50% increase in the tumor: muscle (target:non-target) ratio from 30 min (4.31) to 60 min (8.84) (FIG. 9 inset).

Thus, both microPET and biodistribution studies demonstrate high target binding, specificity and stability of [¹⁸F]KS1.

### Example 11: KS1 does not Generate ROS at Lower Concentrations

To aid in validating KS1 as a PET ROS imaging agent, we demonstrate that KS1 does not act as pro-oxidant, that it does not generate ROS, at lower concentrations (≤1.0 mM). For this, we used dichlorofluorescin diacetate (DCFDA) intracellular ROS detection assay kit and followed manufacture recommended protocol (ZHELEV et al., Docosahexaenoic Acid Sensitizes Leukemia Lymphocytes to Barasertib and Everolimus by ROS-dependent Mechanism Without Affecting the Level of ROS and Viability of Normal Lymphocytes. Anticancer Research 2016, 36 (4), 1673-1682). We studied two patient-derived tumor cell lines: SCC-61, a genetically matched radiation sensitive cell line, and PC3, prostate cancer cell line. The maximum allowed mass for a typical clinical PET radiotracer is <100 μM (Schwarz et al., Radiochemistry and radiopharmacology. LIC-Nuclear Medicine and PET/CT: Technology and Techniques 2016, 11, 77), so we chose 10-100 μM as our concentration range for tracer-level assays. The lowest reported pharmacologic dose for ascorbate is 2.0 mM (Levine et al., Vitamin C: a concentration-function approach yields pharmacology and therapeutic discoveries. Adv Nutr 2011, 2 (2), 78-88), so we chose this dose for our pharmacological assays. We chose the ligand incubation times based on the maximum tracer uptake time (˜2 h) for a typical [¹⁸F]-based PET radiotracer injection (Schlyer, PET tracers and radiochemistry. ANNALS-ACADEMY OF MEDICINE SINGAPORE 2004, 33 (2), 146-154).

The data was expressed as mean fluorescent values±standard deviation (SD) for 6 replicates from each condition (FIG. 10). KS1 at concentrations of 10 μM and 100 μM did not generate ROS in both SCC-61 and PC3 cell lines. There was no significant change in the intensity between KS1 and ascorbate at both the concentrations and in both the tumor cell lines. Fluorescence was measured at hourly interval for 7.0 h, and no change was observed.

The results demonstrate that KS1 does not generate any intracellular ROS up to 100 μM concentrations.

### Example 12: KS1 Generates ROS at Pharmacological Doses

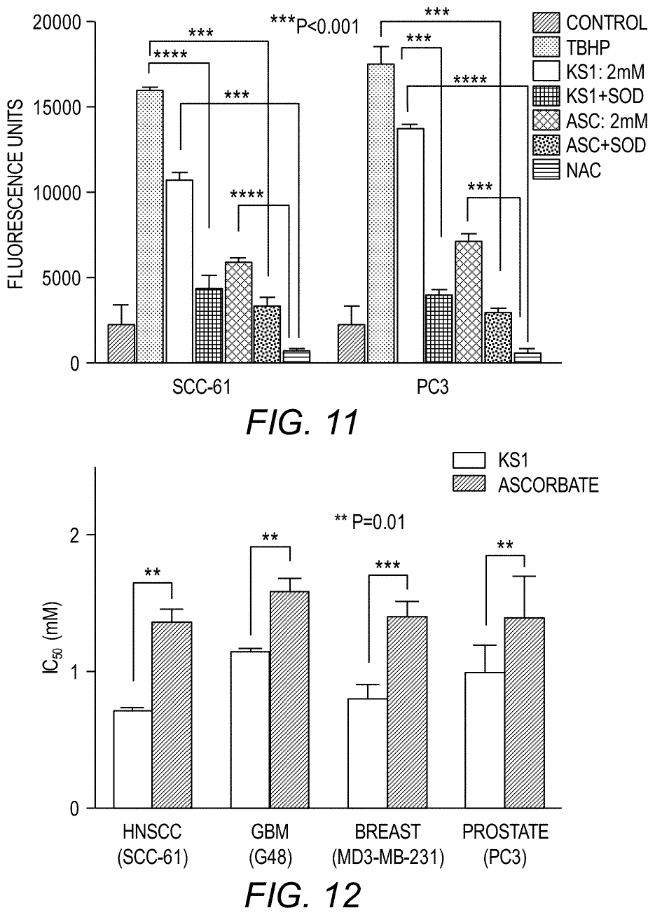

Pharmacologic doses of ascorbate generates ROS, including H₂O₂ and trace amounts of labile iron extracellularly and/or intracellularly (Nauman et al., Systematic Review of Intravenous Ascorbate in Cancer Clinical Trials. Antioxidants (Basel) 2018, 7 (7), 89). We performed the same DCFDA assay with KS1 and ascorbate at pharmacological doses of 2 mM for 7 h in both SCC-61 and PC3 cell lines. ROS production was analyzed and reported (FIG. 11) using the similar measurements as above. Ligand specificity is commonly determined by pretreating the cells with high concentrations of blockers. We used superoxide dismutase (SOD), an enzyme that selectively suppresses accumulation of superoxide peroxide anions (Bielski et al., Chemistry of ascorbic acid radicals. in Ascorbic Acid: Chemistry, Metabolism, and Uses. Chapter 1982, 4, 81-100).

Fluorescence intensity with KS1 at 2 mM increased significantly compared to the intensity at 100 μM. The fluorescence intensity was close to the positive control, TBHP. More importantly, the increased intensity was subdued (˜3-fold) with SOD addition. Fluorescent intensity with ascorbate also increased, but not as with KS1. Most of the literature on ascorbate is focused on its cytotoxic mechanisms (Alexander et al., Pharmacologic Ascorbate Reduces Radiation-Induced Normal Tissue Toxicity and Enhances Tumor Radiosensitization in Pancreatic Cancer. Cancer Research 2018, 78 (24), 6838; Chen et al., Ascorbate in pharmacologic concentrations selectively generates ascorbate radical and hydrogen peroxide in extracellular fluid. Proceedings of the National Academy of Sciences 2007, 104 (21), 8749); Ohno et al., High-dose Vitamin C (Ascorbic Acid) Therapy in the Treatment of Patients with Advanced Cancer. Anticancer Research 2009, 29 (3), 809-815).

The data suggests that KS1 induces ROS generation at higher/pharmacological doses similar to ascorbate, and blocking data suggest that KS1 generates primarily superoxide peroxide anions as ROS, similar to ascorbate.

### Example 13: Cytotoxicity in Tumor Cells at Pharmacologic Doses, MTT IC₅₀ Assays

There is growing evidence for a therapeutic role of ascorbate against several cancers (Creagan et al., Failure of High-Dose Vitamin C (Ascorbic Acid) Therapy to Benefit Patients with Advanced Cancer. New England Journal of Medicine 1979, 301 (13), 687-690; Chen et al., Pharmacologic ascorbic acid concentrations selectively kill cancer cells: action as a pro-drug to deliver hydrogen peroxide to tissues. Proceedings of the National Academy of Sciences of the U.S. Pat. No. 2,005,102 (38), 13604-13609; Prasad et al., High Doses of Multiple Antioxidant Vitamins: Essential Ingredients in Improving the Efficacy of Standard Cancer Therapy. Journal of the American College of Nutrition 1999, 18 (1), 13-25). Pharmacologic doses of ascorbate (oral and/or intravenous) could increase the effectiveness of cancer chemotherapy in many solid cancer therapies (López-Lázaro, Dual role of hydrogen peroxide in cancer: Possible relevance to cancer chemoprevention and therapy. Cancer Letters 2007, 252 (1), 1-8; Li et al., New Developments and Novel Therapeutic Perspectives for Vitamin C. The Journal of Nutrition 2007, 137 (10), 2171-2184; Klimant et al., Intravenous vitamin C in the supportive care of cancer patients: a review and rational approach. Current Oncology 2018, 25 (2), 139-148. The primary objective of this experiment was to determine the cytotoxic effects of KS1 at pharmacological concentrations. Cancer cell lines were selected based on their high ROS implications. We performed cytotoxicity tests of KS1 and ascorbate using MTD assays (96-well plate assay) (He et al., The changing 50% inhibitory concentration (IC(50)) of cisplatin: a pilot study on the artifacts of the MTT assay and the precise measurement of density-dependent chemoresistance in ovarian cancer. Oncotarget 2016, 7 (43), 70803-70821; Sobottka et al., Assessment of antineoplastic agents by MTT assay: partial underestimation of antiproliferative properties. Cancer chemotherapy and pharmacology 1992, 30 (5), 385-393) in patient-derived cancer cell lines of HNSCC (SCC-61), glioblastoma (G48), breast cancer (MD3-MB-231), and prostate cancer (PC3) using the MTT assay. These cell lines have been studied extensively with ascorbic acid treatments (Schoenfeld et al., O(2)(.-) and H(2)O(2)-Mediated Disruption of Fe Metabolism Causes the Differential Susceptibility of NSCLC and GBM Cancer Cells to Pharmacological Ascorbate. Cancer Cell 2017, 31 (4), 487-500.e8; Chen et al., Modulators of Redox Metabolism in Head and Neck Cancer. Antioxidants & Redox Signaling 2017, 29 (16), 1660-1690; Khandrika et al., Oxidative stress in prostate cancer. Cancer Lett 2009, 282 (2), 125-136.

IC₅₀ values were presented as mean±SD from at least three independent experiments (FIG. 12). IC₅₀ of KS1 and ascorbate in SCC-61 was 0.718 mM and 1.380 mM, respectively. In PC3 cells, KS1 and ascorbate displayed IC₅₀ values of 1.01 mM and 1.41 mM, respectively.

Importantly, KS1 showed similar or slightly higher potency than ascorbate in all cancer cell lines tested, with >1.6 to 2.5-fold selectivity in SCC-61, PC3, and MD3-MB-231 cells. Though not considered a primary criterion for PET imaging probe selection, we evaluated IC₅₀ values of KS1 with ascorbate as reference standard from 0.1 mM to 2 mM, using MTT assays and controls. This aids in identifying the concentration switch from its ROS-imaging to possible tumor-killing roles.

### Example 14: [¹⁸F]KS1 In Vitro Cell Uptake Assay in HNSCC Cell Line

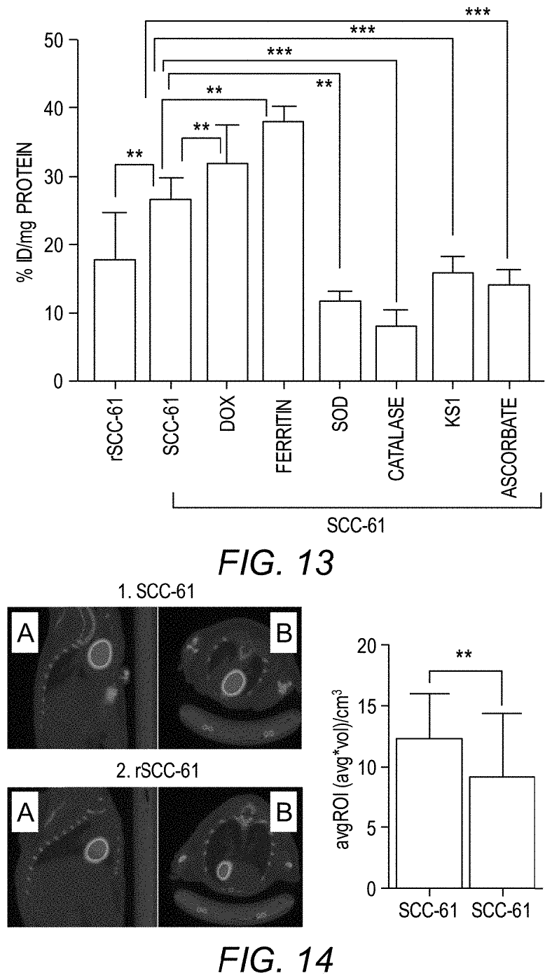

We reported the basic in vitro cell uptake assay of [¹⁸F]KS1 in PC3 and SCC-61 cell lines using KS1 and ascorbate as blockers (Solingapuram Sai et al., Initial biological evaluations of 18F-KS1, a novel ascorbate derivative to image oxidative stress in cancer. EJNMMI Research 2019, 9 (1), 43), following previously published protocols by our group (Sai, et al., Peptide-based PET imaging of the tumor restricted IL13RA2 biomarker. Oncotarget 2017, 8 (31), 50997-51007; Solingapuram Sai et al., Radiolabeling and initial biological evaluation of [(18)F]KBM-1 for imaging RAR-α receptors in neuroblastoma. Bioorganic & medicinal chemistry letters 2017, 27 (6), 1425-1427; Sattiraju et al., IL13RA2 targeted alpha particle therapy against glioblastomas. Oncotarget 2017, 8 (26), 42997-43007). Here we report complete cell uptake of [¹⁸F]KS1 in SCC-61 and rSCC-61 cell lines of HNSCC using several ROS blocking agents including ascorbate, SOD, catalase, DHE, and non-radioactive compounds KS1 (100 μM) and few ROS inducers including doxorubicin and ferritin. These agents were added in the cell media to the seeded tumor cells 1.0 h prior to radiotracer addition (n=6 per blocker). The γ counts per minute values of each well were normalized to the amount of radioactivity added to each well and the protein concentration in the well and expressed as percent uptake relative to the control condition. The data were expressed as % ID/mg of protein present in each well. SCC-61 cells have higher oxidative stress with higher intracellular ROS and protein oxidation than rSCC-61 (Sai et al., Peptide-based PET imaging of the tumor restricted IL13RA2 biomarker. Oncotarget 2017, 5; Yamamoto et al., Correlation of 18F-FLT and 18F-FDG uptake on PET with Ki-67 immunohistochemistry in non-small cell lung cancer. European Journal of Nuclear Medicine and Molecular Imaging 2007, 34 (10), 1610-1616; Solingapuram Sai et al., Radiolabeling and initial biological evaluation of [18F]KBM-1 for imaging RAR-α receptors in neuroblastoma. Bioorganic & Medicinal Chemistry Letters 2017, 27 (6), 1425-1427).

[¹⁸F]KS1 radioactive uptake in SCC-61 cells was ˜3-fold higher compared to rSCC-61 uptake (FIG. 13). Importantly, the uptake in SCC-61 cells was ˜65%, ˜70%, and ˜65% blocked by SOD, nonradioactive KS1, and ascorbate respectively.

Catalase is the common enzyme that catalyzes the decomposition of H₂O₂ to water and oxygen. When catalase was added, the radioactive uptake was inhibited by ˜90% suggesting that [¹⁸F]KS1 might specifically target hydrogen peroxide-based ROS.

Doxorubicin (Dox) is a highly effective anticancer drug that is frequently employed to treat hematological and solid tumors which primarily acts by formation of superoxide peroxide (Chu et al., Development of a PET Radiotracer for Noninvasive Imaging of the Reactive Oxygen Species, Superoxide, in vivo. Organic & biomolecular chemistry 2014, 12 (25), 4421-4431; Robb et al., Selective superoxide generation within mitochondria by the targeted redox cycler MitoParaquat. Free Radical Biology and Medicine 2015, 89, 883-894; Bansal et al., Broad Phenotypic Changes Associated with Gain of Radiation Resistance in Head and Neck Squamous Cell Cancer. Antioxidants & Redox Signaling 2014, 21 (2), 221-236; Halasi et al., ROS inhibitor N-acetyl-1-cysteine antagonizes the activity of proteasome inhibitors. The Biochemical journal 2013, 454 (2), 201-208; Che et al., Expanding roles of superoxide dismutases in cell regulation and cancer. Drug discovery today 2016, 21 (1), 143-149; Ryan et al., Reactive Oxygen and Nitrogen Species Differentially Regulate Toll-Like Receptor 4-Mediated Activation of NF-κB and Interleukin-8 Expression. Infection and Immunity 2004, 72 (4), 2123-2130; Costa et al., Dipyrone and aminopyrine are effective scavengers of reactive nitrogen species. Redox Report 2006, 11 (3), 136-142; Azad et al., Ebselen induces reactive oxygen species (ROS)-mediated cytotoxicity in Saccharomyces cerevisiae with inhibition of glutamate dehydrogenase being a target( ). FEBS Open Bio 2014, 4, 77-89). Iron-storage protein, ferritin stimulates ascorbate production through formation of ROS (Che et al., Expanding roles of superoxide dismutases in cell regulation and cancer. Drug discovery today 2016, 21 (1), 143-149). Treatment with ROS-inducer Dox increased the radioactive uptake by ˜20% and ferritin by ˜32%. The increase in uptake was not as high as expected, but that could relate to (a) the in vitro nature of the Dox assay and (b) timing (and dosages) of our Dox incubations.

Cell uptake studies suggest some degree of nonspecific binding, commonly associated with routine and novel radiotracer evaluations in cancer. This is likely due to in vitro artifacts or not completely saturating the receptor sites. These in vitro cell uptake data with different blockers serve as a gain-of-function/proof-principle (Wagner et al., Approaches using molecular imaging technology—use of PET in clinical microdose studies. Adv Drug Deliv Rev 2011, 63 (7), 539-546; Sai et al., 18F-AFETP, 18F-FET, and 18F-FDG imaging of mouse DBT gliomas. J Nucl Med 2013, 54 (7), 1120-1126) of the specificity of KS1. Ascorbate pre-addiction significantly blocked the uptake. Ascorbate increases the concentration of the iron storage protein, ferritin (Schoenfeld et al., O(2)(.-) and H(2)O(2)-Mediated Disruption of Fe Metabolism Causes the Differential Susceptibility of NSCLC and GBM Cancer Cells to Pharmacological Ascorbate. Cancer Cell 2017, 31 (4), 487-500.e8; Alexander et al., Pharmacological ascorbate reduces radiation-induced normal tissue toxicity and enhances tumor radiosensitization in pancreatic cancer. Cancer Research 2018, canres.1680.2018); a similar increase was also seen with our [¹⁸F]KS1.

This suggests that the binding properties of [¹⁸F]KS1, including ROS interactions might be similar to ascorbate. Strong in vitro cell uptake data demonstrate superior binding of [¹⁸F]KS1 towards peroxide-based ROS processes.

### Example 15: In Vivo MicroPET/CT Imaging

After evaluating the complete in vitro ROS uptake and stability profile of [¹⁸F]KS1, we performed in vivo microPET/CT scans in tumor-bearing mice. To test our hypothesis of selective binding of [¹⁸F]KS1 to image ROS in tumor tissue, we reported the baseline and blocking PET imaging data in a murine model of PCa (Solingapuram Sai et al., Initial biological evaluations of 18F-KS1, a novel ascorbate derivative to image oxidative stress in cancer. EJNMMI Research 2019, 9 (1), 43). Further, to test the differential ROS uptake of [¹⁸F]KS1, microPET/CT imaging experiments were performed in both SCC-61 and rSCC-61 tumor-bearing mice (n=5, 25±2.5 g).

From basic region of interest (ROI) analysis on PET scans, ROS-expressing SCC-61 tumors showed ˜1.2-fold higher tumor uptake than rSCC-61 tumors (FIG. 14), demonstrating moderate target tissue selectivity. More importantly, no residual uptake was seen in other organs, including bladder, and kidney, post 1-h radiotracer injection.

### Example 16: Ex Vivo Biodistribution Studies

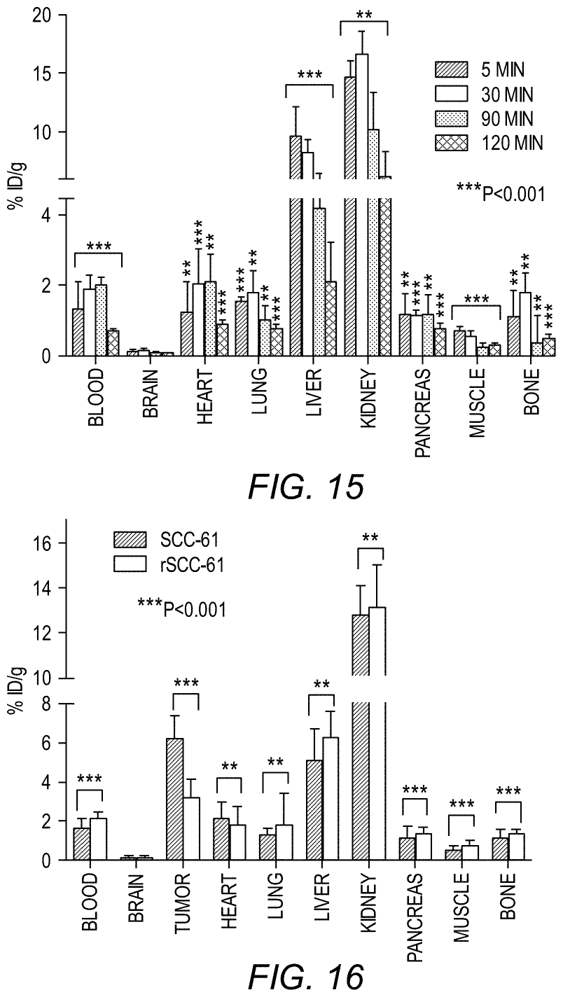

To determine the accumulation and clearance pattern of [¹⁸F]KS1, we first evaluated its biodistribution in normal ICR mice (n=8; 4 male and 4 female mice, 22±5 g) at 5, 30, 90 and 120 min post-radiotracer injection (FIG. 15). [¹⁸F]KS1 displayed rapid clearance from most major peripheral organs from 5 min to 120 min post injection i.e., blood from 5 min (1.361±0.71) to 120 min (0.31±0.071), liver from 5 min (9.72±2.55) to 120 min (2.124±1.12) and kidney from 5 min (14.793±1.33) to 120 min (3.23±1.30) post-injection. No significant bone uptake was seen, indicating no defluorination of the radiotracer. Poor brain uptake indicates a limitation of [¹⁸F]KS1 to track ROS in brain when administered in this manner. Favorable pharmacokinetics of [¹⁸F]KS1, including rapid washout of activity from peripheral organs, renal and/or hepatic clearance, and non-significant bone uptake suggest translational utility.

Subsequently, post-PET biodistribution was performed in the same SCC-61 or rSCC-61 tumor-bearing mice injected with [¹⁸F]KS1 (FIG. 16). The distribution results show excellent target (tumor) to non-target (muscle) ratio in SCC-61 (10.93) and rSCC-61 tumors (4.11), with ˜2.5-fold enrichment in SCC-61 tumors. High kidney and liver uptake were common with [¹⁸F] radiotracer's distribution at 30 min post-injection.

These biodistribution results were consistent with in vitro cell uptake and microPET/CT imaging results. [¹⁸F]KS1's higher tumor uptake in SCC-61 tumors establishes its validity to clearly distinguish high from low ROS tumor tissue. Importantly, the radioactivity profile in other (non-tumor) organs is similar to distribution kinetics in normal mice at 30 min post-injection.

Taken together, these in vivo and ex vivo findings suggest that [¹⁸F]KS1 exhibited high tumor uptake in vivo, with superior specificity and selectivity in both HNSCC and PCa tumor-bearing mice, and favorable pharmacokinetics, with excellent washout from peripheral organs.

The foregoing is illustrative of the present invention, and is not to be construed as limiting thereof. The invention is defined by the following claims, with equivalents of the claims to be included therein.

That which is claimed is:

1. A compound of Formula (I): 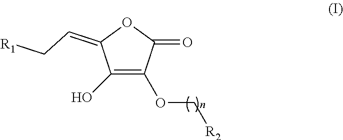
wherein:
R₁ is a substituted aryl or substituted heteroaryl, which is substituted with —X(CH₂)ₘ—R₃,
wherein X is O, S, NH, or CH₂;
m is 0, 1, 2, 3, 4, 5, or 6;
R₃ is:
(i) an imaging moiety selected from the group consisting of ¹⁸F, ⁷⁶Br, ¹²³I, and ¹¹C;
(ii) a leaving group selected from the group consisting of a halo (e.g., Cl, F, Br and/or I) and a sulfonate (e.g., triflate, mesylate, tosylate, brosylate and/or nosylate); or
(iii) a chelator moiety (e.g., DOTA, NOTA and DFO) associated with a radioisotope selected from ⁶⁸Gd, ⁶⁴Cu, and ⁸⁹Zr;
n is 0; and
R₂ is H;
or wherein:
R₁ is halo (e.g., Cl, Br, F, I);
n is 0, 1, 2, 3, 4, 5, or 6; and
R₂ is a substituted aryl or substituted heteroaryl, which is substituted with —X(CH₂)ₘ—R₃,
wherein X is O, S, NH or CH₂;
m is 0, 1, 2, 3, 4, 5, or 6;
R₃ is:
(i) an imaging moiety selected from the group consisting of ¹⁸F, ⁷⁶Br, ¹²³I, and ¹¹C;
(ii) a leaving group selected from the group consisting of a halo (e.g., Cl, F, Br and/or I) and a sulfonate (e.g., triflate, mesylate, tosylate, brosylate and/or nosylate); or
(iii) a chelator moiety (e.g., DOTA, NOTA and DFO) associated with a radioisotope selected from ⁶⁸Gd, ⁶⁴Cu, and ⁸⁹Zr;
or a pharmaceutically acceptable salt thereof.

2. The compound of claim 1, wherein said compound is a compound of Formula (I)(a): 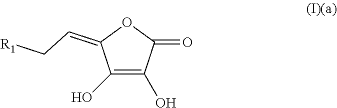
wherein:
R₁ is a substituted aryl or substituted heteroaryl, which is substituted with —X(CH₂)ₘ—R₃,
wherein X is O, S, NH or CH₂;
m is 0, 1, 2, 3, 4, 5, or 6; and
R₃ is: (i) an imaging moiety selected from the group consisting of ¹⁸F and ¹¹CH₃; or (ii) a leaving group selected from the group consisting of F and a sulfonate (e.g., a tosylate);
or a pharmaceutically acceptable salt thereof.

3. The compound of claim 1, wherein the compound is a compound of Formula (I)(b): 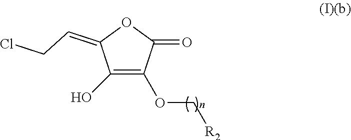
wherein
n is 1;
R₂ is a substituted aryl or substituted heteroaryl, which is substituted with —X(CH₂)ₘ—R₃,
wherein X is O, S, NH or CH₂;
m is 0, 1, 2, 3, 4, 5, or 6; and
R₃ is: (i) an imaging moiety selected from the group consisting of ¹⁸F and ¹¹CH₃; or (ii) a leaving group selected from the group consisting of F and a sulfonate (e.g., a tosylate);
or a pharmaceutically acceptable salt thereof.

4. The compound of claim 2, wherein R₁ is a substituted aryl selected from anthracenyl, azulenyl, fluorenyl, indanyl, indenyl, naphthyl, phenyl, and tetrahydronaphthyl.

5. The compound of claim 3, wherein R₂ is a substituted aryl selected from the group consisting of anthracenyl, azulenyl, fluorenyl, indanyl, indenyl, naphthyl, phenyl, and tetrahydronaphthyl.

6. The compound of claim 4, wherein the substituted aryl is a substituted phenyl.

7. The compound of claim 2, wherein R₁ is a substituted heteroaryl selected from the group consisting of benzoxadiazolyl, benzoxazolyl, benzofuranyl, benzothienyl, furanyl, imidazolyl, indazolyl, indolyl, isoxazolyl, isoquinolinyl, isothiazolyl, naphthyridinyl, oxadiazolyl, oxazolyl, pyridinyl, pyridazinyl, pyrimidinyl, pyrazinyl, pyrazolyl, pyrrolyl, quinolinyl, thiazolyl, thienopyridinyl, thienyl, triazolyl, thiadiazolyl, and triazinyl.

8. The compound of claim 3, wherein R₂ is a substituted heteroaryl selected from benzoxadiazolyl, benzoxazolyl, benzofuranyl, benzothienyl, furanyl, imidazolyl, indazolyl, indolyl, isoxazolyl, isoquinolinyl, isothiazolyl, naphthyridinyl, oxadiazolyl, oxazolyl, pyridinyl, pyridazinyl, pyrimidinyl, pyrazinyl, pyrazolyl, pyrrolyl, quinolinyl, thiazolyl, thienopyridinyl, thienyl, triazolyl, thiadiazolyl, and triazinyl; and n is 1.

9. The compound of claim 7, wherein the substituted heteroaryl is triazolyl, pyridinyl or pyrrolyl.

10. The compound of claim 1, wherein X is O; m is 2; and R₃ is ¹⁸F, F, or a sulfonate.

11. The compound of claim 1, wherein m is 0 and R₃ is ¹¹CH₃.

12. The compound of claim 1, wherein the compound of Formula (I) is selected from the group consisting of: 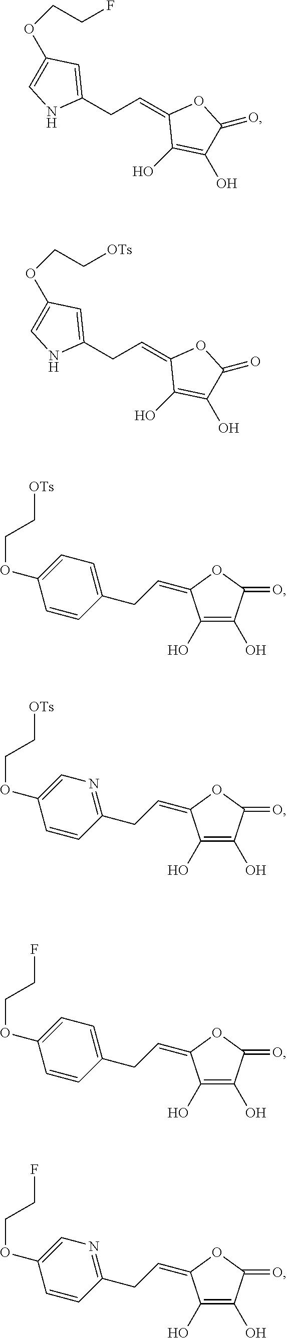 [SMILES: O=C1O/C(=C/Cc2cc(OCCF)c[nH]2)C(O)=C1O ; Cc1ccc(S(=O)(=O)OCCOc2c[nH]c(C/C=C3/OC(=O)C(O)=C3O)c2)cc1 ; Cc1ccc(S(=O)(=O)OCCOc2ccc(C/C=C3/OC(=O)C(O)=C3O)cc2)cc1 ; Cc1ccc(S(=O)(=O)OCCOc2ccc(C/C=C3/OC(=O)C(O)=C3O)nc2)cc1 ; O=C1O/C(=C/Cc2ccc(OCCF)cc2)C(O)=C1O ; O=C1O/C(=C/Cc2ccc(OCCF)cn2)C(O)=C1O] 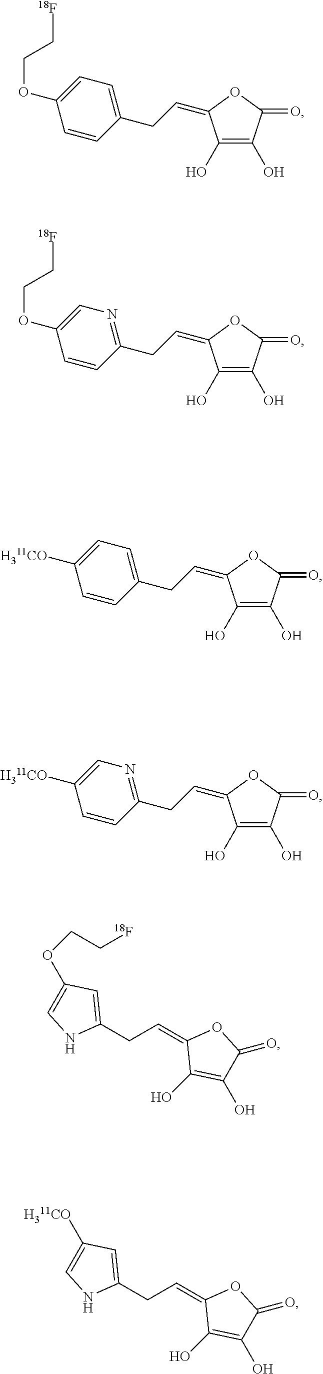 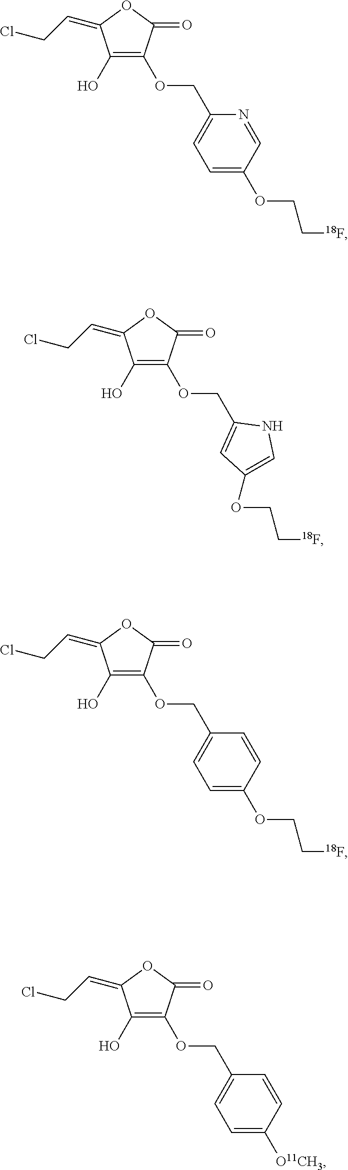 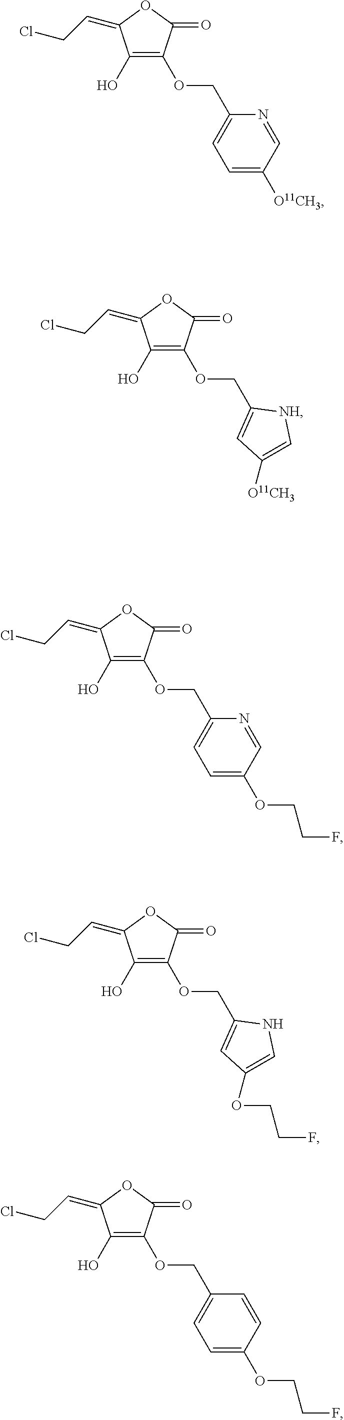 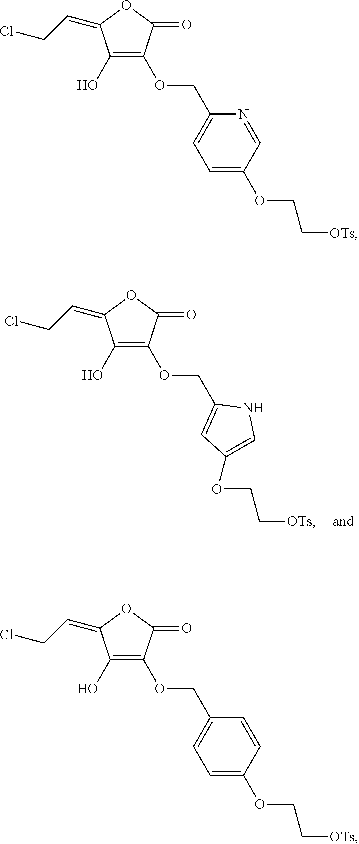 [SMILES: Cc1ccc(S(=O)(=O)OCCOc2ccc(COC3=C(O)/C(=C\CCl)OC3=O)nc2)cc1 ; Cc1ccc(S(=O)(=O)OCCOc2c[nH]c(COC3=C(O)/C(=C\CCl)OC3=O)c2)cc1 ; Cc1ccc(S(=O)(=O)OCCOc2ccc(COC3=C(O)/C(=C\CCl)OC3=O)cc2)cc1]
or a pharmaceutically acceptable salt thereof.

13. The compound of claim 1, wherein R₃ is a ¹⁸F imaging moiety and exhibits a radiochemical specific activity from about 2500 to about 4000 mCi/μmol.

14. The compound of claim 1, wherein the compound exhibits at least 80% serum stability for about 120 minutes post synthesis when contacting human serum.

15. A method of measuring reactive oxygen species (ROS) or imaging oxidative stress in cells comprising:
contacting cells with an effective amount of a compound of claim 1; and
measuring gamma radiation emitted by the compound.

16. The method of claim 15, wherein the cells are cancer cells selected from head and neck squamous cell carcinoma, glioblastoma, breast cancer, and prostate cancer.

17. A method of imaging/detecting/diagnosing a ROS modulated illness comprising:
administering an effective amount of the compound of claim 1 to a subject in need thereof;
detecting gamma radiation emitted by the compound; and
forming an image therefrom.

18. The method of claim 17, wherein the ROS modulated illness is selected from diabetes, cardiovascular diseases, atherosclerosis, hypertension, ischemia, reperfusion injury, neurodegeneration, rheumatoid arthritis, and cancer.

19. A method of making a compound of Formula (I)(b)(i): 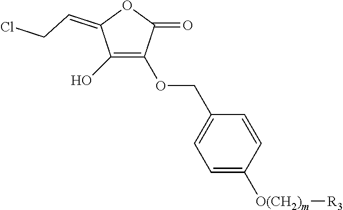
wherein:
m is 0 and R₃ is CH₃; or
m is 1, 2, 3, 4, 5 or 6 and R₃ is a leaving group (e.g., a sulfonate (e.g., tosylate, mesylate, brosylate, nosylate, triflate, besylate) or a halo),
comprising:
(a) providing a compound of Formula (A): 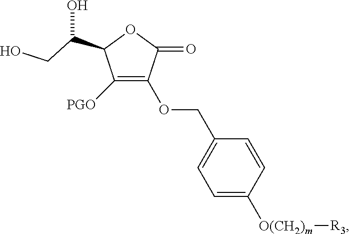
wherein PG is a hydroxyl protecting group, and m is as defined above;
(b) contacting the compound of Formula (A) with a sulfonyl halide (e.g., tosyl chloride) to form a compound of Formula (B): 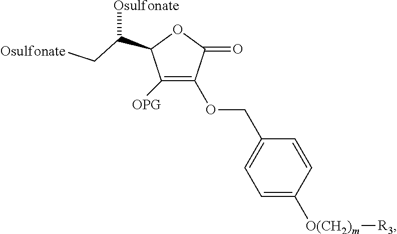
wherein PG, m and R₃ are as defined above;
(c) contacting the compound of Formula (B) with Et₄NCl and a base (e.g., DBU) to form a compound of Formula (C): 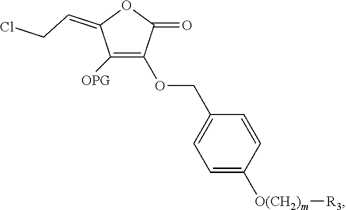
wherein PG, m and R₃ are the same as defined above;
(d) purifying the compound of Formula (C) to obtain the compound of Formula (C) with a chemical purity of at least 90%; and then
(e) removing the protecting group PG of the compound of Formula (C), to obtain the compound of Formula (I)(b)(i).

20. A method of producing a radiotracer compound of Formula I: 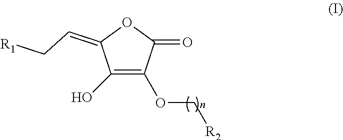
wherein:
R₁ is a substituted aryl or substituted heteroaryl, which is substituted with —X(CH₂)ₘ—R₃,
wherein X is O, NH, S or CH₂;
m is 0, 1, 2, 3, 4, 5, or 6;
R₃ is an ¹⁸F imaging moiety;
n is 0; and
R₂ is H;
or wherein:
R₁ is halo;
n is 0, 1, 2, 3, 4, 5, or 6; and
R₂ is a substituted aryl or substituted heteroaryl, which is substituted with —X(CH₂)ₘ—R₃,
wherein X is O, S, NH or CH₂;
m is 0, 1, 2, 3, 4, 5, or 6; and
R₃ is an ¹⁸F imaging moiety,
or a pharmaceutical acceptable salt thereof;
comprising:
(a) mixing a precursor compound of Formula (I); 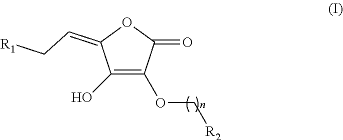
wherein R₁ is a substituted aryl or substituted heteroaryl, which is substituted with —X(CH₂)ₘ—R₃,
wherein X is O, NH, S or CH₂;
m is 0, 1, 2, 3, 4, 5, or 6;
R₃ is a leaving group;
n is 0; and
R₂ is H;
or wherein:
R₁ is halo;
n is 0, 1, 2, 3, 4, 5, or 6; and
R₂ is a substituted aryl or substituted heteroaryl, which is substituted with —X(CH₂)ₘ—R₃,
wherein X is O, S, NH or CH₂;
m is 0, 1, 2, 3, 4, 5, or 6;
R₃ is a leaving group selected from a sulfonate (e.g., tosylate) and halo,
with a solvent and a solution of [¹⁸F]F to obtain a reaction mixture;
(b) heating the reaction mixture obtained in step (a); and then
(c) purifying the reaction mixture, to produce the radiotracer compound.
